(19) United States
(12) Patent Application Publication
(10) Pub. No.: US 2009/0162848 A1
(43) Pub. Date: Jun. 25, 2009

(54) Noxin, a novel stress-induced gene involved in cell cycle and apoptosis

(75) Inventors: Naoki Nakaya, Rockville, MD (US); Jill Hemish, Cold Spring Harbor, NY (US); Grigori Enikolopov, Cold Spring Harbor, NY (US)

Correspondence Address: ROPES & GRAY LLP, PATENT DOCKETING 39/361, 1211 AVENUE OF THE AMERICAS, NEW YORK, NY, 10036-8704, US

(73) Assignee: COLD SPRING HARBOR LABORATORY, Cold Spring Harbor, NY (US)

(21) Appl. No.: 12/228,148

(22) Filed: Aug. 7, 2008

Related U.S. Application Data
(60) Provisional application No. 60/964,077, filed on Aug. 8, 2007.

(51) Int. Cl. C12Q 1/68; C12N 15/11; C12N 15/00; A01K 67/027; C12N 5/06; C12N 15/82; C12Q 1/02

(52) U.S. Cl. 435/6; 536/23.5; 435/320.1; 800/13; 800/14; 435/354; 435/455; 435/29

(57) ABSTRACT

The invention provides for novel noxin polypeptides and nucleic acids from humans, rats, and mice and to related compositions and uses. The invention also provides for the creation of non-human, transgenic mammals which have, incorporated in their genome, DNA that includes a sequence of a mammalian noxin gene that does not produce noxin polypeptides, i.e. a noxin knockout mouse.

The invention also provides for methods for protecting a cell from stress damage by enhancing the expression noxin, methods for decreasing cell death by enhancing the expression of noxin, methods for inducing cell death by decreasing noxin activity, methods for inducing cell cycle arrest by increasing noxin, and methods for preventing cell cycle arrest by inhibiting noxin activity.

### FIELD OF THE INVENTION

This invention relates to a novel conserved gene and its protein product, designated noxin (nitric oxide-inducible gene and its polypeptide, which plays a role in the mammalian cell cycle, minimizing damage to the cell from stressors. This invention also relates to mammals where the expression of one gene has been suppressed. More specifically, the invention concerns insertion of an exogenous DNA construct into the genomic DNA of mammals, thereby producing transgenic mammals with decreased or completely suppressed expression of an endogenous gene.

### BACKGROUND OF THE INVENTION

Cells respond to oxidative and genotoxic stress by withdrawing from the cell cycle, repairing the damaged regions of DNA, repairing or destroying affected proteins, altering the growth characteristics, and seeking to inactivate the stressor. Alternatively, if the stress-induced damage is too extensive, cells may be eliminated by apoptosis. Various stressors (e.g., ionizing radiation, ultraviolet radiation, reactive oxygen and nitrogen species, and alkylating chemicals) act differently and cause distinct types of damage to the cell; at the same time, these dissimilar insults activate shared sets of molecules and pathways aimed at minimizing the damage and repairing the affected cell components (Bakkenist et al., (2004) Cell 118:9-17; Gudkov et al., (2003)Cancer 3:117-29; Harris et al., (2005) Oncogene 24:2899-2908; and Kastan et al., (2004) Nature 432:316-23). The cellular defense mechanisms include immediate responses (e.g., posttranslational modifications of tumor suppressor protein p53, leading to its accumulation in the cells) as well as more extended responses (e.g., transcriptional activation of genes whose products help the cells to complete the repair process or to communicate the stress and repair signals to the surrounding cells). This coordinated set of protein modification and gene activation events helps ensure that the damage to cells is minimized and that cells restore their pre-stress status (e.g., returning back to cycling).

Nitric oxide (NO) is a versatile signaling molecule, which is involved in both physiologic (e.g., vasorelaxation and neurotransmission) and pathologic (e.g., inflammation and cell death) processes in the organism (Boehning et al., (2003) Annu. Rev. Neurosci. 26:105-31; Ignarro, (2000) 1st ed. Academic Press, San Diego; Nathan, (2003) J. Clin. Invest. 111:769-778). When produced at high levels (e.g., by the high-output inducible NOS isoform), it can induce cell damage and subsequent apoptosis (Brüne, (2003) Cell Death Differ. 10:864-869; Li et al., (2005) Cancer Let. 226:1-15). At lower levels, NO can act as an antiproliferative agent in vitro and in vivo, contributing to cell cycle arrest during cell differentiation or inflammation When acting upon the cell cycle machinery, NO affects multiple pathways and can contribute to cell cycle arrest through several independent mechanisms. (Contestabile et al., (2004) Neurochemistry Int. 45:903-914; Enikolopov et al., (1999) Cell Death Differ. 6:956-63; Estrada et al., (2005) Neuroscientist 111:294-307; Gibbs, (2003) Mol. Neurobiol. 27:107-20; Nathan, (2003) J. Clin. Invest. 111:769-778; Packer et al., (2003) Proc. Nat. Acad. Sci. U.S.A. 100:9566-71; and Peunova et al., (1995) Nature 375:68-73). Several major regulators of the cell cycle and stress response, e.g., cyclin-dependent kinase 2 (cdk2), cdk inhibitor p21/WAF, cyclin D1, PCNA, ribonucleotide reductase, mdm2, p53, and ataxia telangectasia mutated kinase (ATM) are involved in the cellular response to NO (Bartek et al., (2001) Curr. Opin. Cell Biol. 13:738-47; Hofseth et al., (2003) Proc. Nat. Acad. Sci. U.S.A. 100: 143-8; McLaughlin et al., (2005) Cancer Res. 65:6097-104; Sharma et al., (1999) Am. J. Physiol. 276:H1450-9; Tanner et al., (2000) Circulation 101: 1982-9). Given the extent of NO involvement in cell physiology, it is likely that specific additional components mediate the response to NO in particular contexts. At the same time, it is conceivable that responses to NO engage mechanisms that are employed by cells in responding to other stressful stimuli.

### SUMMARY OF THE INVENTION

This invention provides for an isolated nucleic acid encoding a novel gene, noxin, so named for its activation: i.e. nitric oxide-inducible gene. One embodiment of the invention is an isolated noxin gene having a nucleotide sequence depicted in SEQ ID NO: 1 (mouse), SEQ ID NO: 3 (human), or SEQ ID NO: 5 (rat) or a fragment thereof. In another embodiment, an isolated nucleic acid of the invention is a naturally occurring variant of the gene depicted as SEQ ID NO: 1, 3, or 5, or a fragment thereof. In another embodiment, an isolated nucleic acid of the invention is a corresponding noxin gene from any organism, or a fragment of such nucleic acid. By “corresponding” it is meant that a gene serves analogous, comparatively the same, function in an organism to which it is endogenous as mouse, human, or rat noxin gene represented respectively by SEQ ID NO: 1, 3, or 5 serves in each of mouse, human or rat. Other embodiments of the invention are genes which sequence is 70, 80, 90, 95, 97, or 99% identical to SEQ ID NO: 1, 3, or 5, or to the fragment thereof. Another embodiment of the invention is an isolated nucleic acid encoding a polypeptide having the amino acid sequence depicted in SEQ ID NO: 2 (mouse), SEQ ID NO: 4 (human) or SEQ ID NO: 6 (rat), or a fragment thereof.

An embodiment of the invention is an isolated nucleic acid complementary to any of the nucleic acid above and fragments thereof.

Another aspect of the invention is an isolated polypeptide having an amino acid sequence depicted in SEQ ID NO: 2 (mouse), SEQ ID NO: 4 (human), or SEQ ID NO: 6 (rat), or a fragment thereof. Other embodiments of the invention are polypeptides of which amino acid sequence is 70, 80, 90, 95, 97, or 99% identical to SEQ ID NO: 2, 4, or 6, or to the fragment thereof.

Another aspect of the invention is a vector comprising the nucleic acid molecule encoding noxin as described above. In one embodiment, the vector is an expression vector. In one embodiment, the vector further comprises an additional coding sequence fused in-frame with a noxin coding sequence, so that the translated polypeptide is a fusion protein. In one embodiment, the fused polypeptide is designed for the ease of detection or purification.

Another aspect of the invention is a vector comprising the nucleic acid encoding a disrupted noxin gene. In a particular embodiment, the vector comprises an isolated nucleic acid comprising a noxin knockout construct comprising a selectable marker sequence flanked by DNA sequences homologous to the endogenous noxin gene, wherein when said construct is introduced into a mouse or an ancestor of said mouse at an embryonic stage, said selectable marker sequence disrupts the endogenous noxin gene in the genome of said mouse such that said mouse exhibits decreased noxin production as compared to a wild type mouse.

Another aspect of the invention is a cultured host cell comprising such a vector. In one embodiment, the host cell of the invention has integrated into a chromosome such a disrupted noxin gene.

Another aspect of the invention is a transgenic mammal whose genome comprises a disruption of the endogenous noxin gene. In particular, a transgenic mammal is produced by disruption of a genomic noxin gene by insertion of a disrupting vector with a selectable marker sequence, and wherein said disruption results in said mouse exhibiting decreased Noxin protein production as compared to a wild-type mouse.

In one embodiment, the transgenic mammal has a homozygous disruption. In a particular embodiment, the homozygous disruption results in a null mutation of the endogenous gene encoding noxin. The transgenic mammal of the invention includes its progeny or embryo.

In a particular embodiment, the mammal is a rodent. More particularly, the rodent is a mouse or a rat.

One embodiment of the invention is a cell from a transgenic mammal as described above. In a particular embodiment, the cell is a mouse embryonic fibroblast. A related embodiment of the invention is a cell line from a transgenic mammal as described above, comprising the noxin knockout construct described above. In a particular embodiment, the cell line is an embryonic stem cell line. In a particular embodiment, the embryonic stem cell line is a mouse cell line.

Yet another aspect of the invention is a method of protecting a cell from stress damage by increasing noxin activity in the cell. In one embodiment, noxin activity is increased by enhancing the mRNA expression of noxin, thus increasing the noxin polypeptide. In another embodiment, noxin activity is increased by activating noxin polypeptide. In certain embodiments, the stress damage is caused by γ-irradiation, UV-irradiation, adriamycin, activated oxygen such as hydrogen peroxide, cytokines, or nitrogen-donors, e.g., S-nitroso-N-acetyl-D,L-penicillamine (SNAP), 1-hydroxy-2-oxo-3-(N-ethyl-2-aminoethyl)-3-ethyl-1-triazene (NOC12), and DETA NONOate (NOC18).

Another aspect of the invention is a method of decreasing cell death caused by stress by increasing noxin activity in the cell. In one embodiment, noxin activity is increased by increasing noxin mRNA expression. In another embodiment, noxin activity is increased by promoting noxin polypeptide translation. In another embodiment, noxin activity is increased by enhancing noxin activation of expressed noxin polypeptide. The stress damage may be caused by any of the stress factors described above. In certain embodiment, noxin activity is inhibited by inhibiting p53 activity.

In yet another aspect, the invention is a method of inducing cell death by inhibiting noxin activity. In one embodiment, noxin activity is inhibited by inhibiting noxin mRNA expression. In another embodiment, noxin activity is inhibited by inhibiting noxin polypeptide translation. In another embodiment, noxin activity is inhibited by inhibiting noxin activation of expressed noxin polypeptide.

A different aspect of the invention is a method of preventing cell death by increasing noxin activity in the cell. In one embodiment, noxin activity is increased by increasing noxin mRNA expression. In another embodiment, noxin activity is increased by promoting noxin polypeptide translation. In another embodiment, noxin activity is increased by enhancing noxin activation of expressed noxin polypeptide.

Another aspect of the invention is a method of inducing cell cycle arrest by increasing noxin activity. In one embodiment, noxin activity is increased by increasing noxin mRNA expression. In another embodiment, noxin activity is increased by promoting noxin polypeptide translation. In another embodiment, noxin activity is increased by enhancing noxin activation of expressed noxin polypeptide.

Another aspect of the invention is a method of preventing cell cycle arrest by decreasing noxin activity. In one embodiment, noxin activity is inhibited by inhibiting noxin mRNA expression. In another embodiment, noxin activity is inhibited by inhibiting noxin polypeptide translation. In another embodiment, noxin activity is inhibited by inhibiting noxin activation of expressed noxin polypeptide.

Another aspect of the invention is a method of evaluating damage of the genome in a cell by detecting levels of noxin expression in the cell. In one embodiment, the expression level is noxin mRNA quantity. In another embodiment, the expression level is noxin polypeptide quantity.

Yet another aspect of the invention is a method of screening for an agent that affects cellular responses to stress induced damages. In one embodiment, a candidate agent is administered to cells or to an animal under a stress condition or under NO inducing conditions, and the agent's effect on expression of noxin is determined.

In one aspect, the invention is a method for producing a non-human transgenic mammal comprising the steps of:

a) introducing into a fertilized egg of a non-human mammal DNA comprising a disrupted mammalian noxin gene and a reporter gene or a selective marker;

b) introducing said fertilized egg into an oviduct of a non-human mammal of the same species as the source of said fertilized egg to allow said fertilized egg to develop into a viable non-human transgenic mammal; and

c) selecting a non-human transgenic mammal that expresses said reporter or selective marker.

### BRIEF DESCRIPTION OF THE FIGURES

FIG. 1A is a panel of microarray images showing noxin induction by NO donor SNAP. Panels 1B-G show magnified view of the microarray for samples treated with SNAP (B, C), NO12 (D, E), and NO18 (F, G).

FIG. 2A-B shows the deduced amino acid sequence of noxin from mouse, human and rat (panel A) and a schematic arrangement of protein motifs in the noxin polypeptide (panel B).

FIG. 3A-D shows tissue distribution of noxin mRNA.

FIG. 4A-F shows the expression of noxin protein in the testis of the noxin^{+/+} wild type, noxin^{+/-} heterozygote, and noxin^{+/+} homozygote transgenic noxin knockout animals.

FIG. 5A-G shows the subcellular distribution of the transfected noxin protein by fluorescent micrograph.

FIG. 6A-D shows the extent of noxin induction by a variety of stress stimuli as a mRNA by Northern blot image and its quantification (panels A and B), and as a protein by a Western blot image and its quantification (panels C and D),

FIG. 7A-B shows regulation of noxin mRNA by cell cycle

FIG. 8 shows the amounts of noxin protein in by phases of cell cycle by western blot.

FIG. 9 shows the levels of FLAG-noxin in cultured cells by western blot in response to various agents.

FIG. 10 shows the cell cycle distribution of stress-exposed NIH3T3 cells.

FIG. 11A-C shows G₁ cell cycle arrest is induced by ectopic expression of noxin.

FIG. 12A-C shows the efficiency and breakdown of transfection. Panel A shows the ratio of transfected to untransfected cells among various cell populations. Panels B and C show the cell cycle distributions of cells transfected and untransfected by noxin and by green fluorescent protein respectively, as determined by flow cytometry.

FIG. 13A-C shows cells transfected with pFLAG-noxin and the expression of p53.

FIG. 14A-D shows analysis of the p 53 amount in cells by flow cytometry.

FIG. 15 is a schematic drawing showing the strategy for targeted inactivation of the noxin gene.

FIG. 16A shows the analysis by PCR and Southern blot of genomic DNA of transgenic noxin^{-/-} knockout mice. FIG. 16B shows the lack of mRNA production in homozygous noxin^{-/-} mice and reduced production by heterozygous (noxin^{+/-}) compared to wildtype.

FIGS. 17A-L shows the characteristics of mouse embryonic fibroblasts (MEFs) isolated from noxin^{-/-} mice, indicating normal growth and response to SNAP treatment but increased cell death.

FIG. 18A-L is the quantification of the clusters identified in FIG. 17A-L.

FIG. 19A-D shows the characteristics of MEFs isolated from noxin^{-/-} mice and comparing to wild type control, indicating the noxin null cells' normal growth and response to SNAP treatment but increased cell death. Panels 19A and 19B show the distribution of annexin-V-positive cells, Panel 19C shows the % of annexin-V-positive cells under stress conditions, and Panel 19D shows TUNEL positive cells, respectively.

FIG. 20A-B shows the effect of nocodazole treatment on noxin knockdown by various small hairpin RNA interference molecules (shRNA; HPA through HPF) to increase the fraction of apoptotic cells. Panel A shows protein amount by western blot Panel B shows cell cycle phase breakdown.

FIG. 21A-D shows cells that were co-transfected with the HPC or HPF shRNA constructs together with YFP-actin construct and stained with Hoechst.

FIG. 22 is a schematic of the noxin gene and positions of sequences used for the RNAi technique.

FIG. 23 is a schematic of shRNA-containing vector and the dsRNA.

### DETAILED DESCRIPTION OF THE INVENTION

### Overview

This application provides for a novel, stress-induced gene, noxin, and a knockout mouse line with an inactivated noxin gene. Noxin gene does not have sequelogs in the genome. The term ‘sequelog’ denotes a nucleotide or amino acid sequence that is similar, to a specified extent, to another sequence, and does not require evolutionary or functional similarities. Having no sequelog means, within the genome of that organism, there is no other gene with a high sequence similarity with the noxin gene. Noxin gene encodes a highly serine-rich protein with predicted phosphorylation sites for ATM, Akt, and DNA-PK kinases, nuclear localization signals, and a Zn-finger domain. Noxin mRNA and protein levels are under tight control by the cell cycle.

Noxin, identified as a nitric oxide (NO)-inducible gene, is strongly induced by a wide range of stress signals: γ-irradiation, UV-irradiation, hydrogen peroxide, adriamycin, and cytokines; this induction is dependent on p53. Noxin accumulates in the nucleus in response to stress and, when ectopically expressed, noxin arrests the cell cycle at G₁ and acts to counteract apoptotic stimuli, such that its loss results in an increased level of cell death. Although noxin also induces p53, the cell cycle arrest function of noxin is independent of p53 activity. Conversely, noxin mRNA and protein expression is controlled by the cell cycle; it can also be activated by a wide range of stress stimuli.

Noxin is a part of the machinery that protects cells during stress, helping them to withdraw from the cell cycle and repair the damage and opposes apoptosis, and thus directly participate in the repair process, including being a part of the protein complexes that restore the integrity of the genome after insult and stress.

Noxin plays an anti-apoptotic role, such that when noxin gene is inactivated or down-regulated, cells exhibit significantly higher levels of apoptosis than their counterparts with normal levels of noxin. This is evident under basal conditions and is maintained when cells are challenged by a range of dissimilar stress stimuli. Thus, our data suggest that noxin is a general stress-response gene whose expression is related to the cell cycle, and whose product may be important to prevent cell death under a wide range of conditions.

The noxin knockout mice are viable and fertile; however, they have an enlarged heart, several altered hematopoietic parameters, and a decreased number of spermatids. Importantly, loss or down-regulation of noxin leads to increased cell death.

### DEFINITIONS

For convenience, certain terms employed in the specification, examples, and appended embodiments are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article, unless context clearly indicates otherwise. By way of example, “an element” means one element or more than one element.

The term “or” is used herein to mean, and is used interchangeably with, the term “and/or”, unless context clearly indicates otherwise.

The term “including” is used herein to mean, and is used interchangeably with, the phrase “including but not limited to”.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques including protein cleavage of whole antibodies and recombinant expression of fragments and the fragments screened for utility and/or interaction with a specific epitope of interest. Thus, the term includes segments of proteolytically-cleaved or recombinantly-prepared portions of an antibody molecule that are capable of selectively reacting with a certain protein. For example, F(ab)₂ fragments can be generated by treating antibody with pepsin. The resulting F(ab)₂ fragment can be treated to reduce disulfide bridges to produce Fab fragments. In preferred embodiments, the antibody further comprises a label attached thereto and able to be detected, (e.g., the label can be a radioisotope, fluorescent compound, enzyme, or enzyme co-factor). The term antibody also includes polyclonal, monoclonal, or other purified preparations of antibodies and recombinant antibodies.

A “chimeric polypeptide” or “fusion polypeptide” is a fusion of a first amino acid sequence with a second amino acid sequence where the first and second amino acid sequences are not naturally present in a single polypeptide chain.

The term “detection”, in addition to art-recognized meanings, means any process of observing a marker, in a biological sample, whether or not the marker is actually detected. In other words, the act of probing a sample for a marker is a “detection” even if the marker is determined to be not present or below the level of sensitivity. Detection may be a quantitative, semi-quantitative or non-quantitative observation.

An “expression construct” is any recombinant nucleic acid that includes an expressible nucleic acid and regulatory elements sufficient to mediate expression in a suitable host cell. For example, an expression construct may contain a promoter or other RNA polymerase contact site, a transcription start site or a transcription termination sequence. An expression construct for production of a protein may contain, for example a translation start site, such as an ATG codon, a ribosome binding site, such as a Shine-Dalgarno sequence, or a translation stop codon.

The term “isolated” as used in reference to nucleic acids or polypeptides means a nucleic acid or polypeptide, such as an noxin nucleic acid or polypeptide, that is removed from its natural context. For example, an “isolated” polypeptide may be substantially free of other proteins that are normally associated with it. As another example, an “isolated” nucleic acid may be removed from its normal genomic context and recombined with other nucleic acids, such as a cloning vector.

A “knock-out” of a gene means an alteration in the sequence of the gene that results in a decrease of function of the target gene, preferably such that target gene expression is undetectable or insignificant. For example, a knock-out of an endogenous noxin gene means that function of the endogenous noxin gene has been substantially decreased or entirely stopped. “Knock-out” transgenics can be transgenic animals having a heterozygous knock-out of the noxin gene or a homozygous knock-out of the noxin gene. “Knock-outs” also include conditional knock-outs, where alteration of the target gene can occur upon, for example, exposure of the animal to a substance that promotes target gene alteration, introduction of an enzyme that promotes recombination at the target gene site, or other method for directing the target gene alteration postnatally. “Knock-down” is used when the gene expression is reduced by not completely eliminated. “Knock-in” may be used when a gene is transfected to increase (when there is an endogenous copy) or effectuate the expression of the gene.

As used herein, the term “transgenic animal” means a non-human animal, usually a mammal (e.g., mouse, rat, rabbit, hamster, etc.), having a non-endogenous nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal. The term includes the newly born, young offsprings, developing adults, or embryos of the non-human transgenic mammal, as well as newly born, young offsprings, developing adults or embryos of a progeny of the transgenic animal. A term for a sub-group of transgenic animals is “non-human transgenic mammal;” examples of which include mouse, rat, dog, monkey, as well as any other suitable non-human mammalian species. A preferred mammal is a rodent, including a mouse.

The term “nucleic acid” includes, in addition to any art recognized meaning, polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. By analogs it is meant that the nucleic acid includes chemically-modified nucleotides and/or unnatural nucleotides that are not unmodified adenine, thymidine, uracil, cytosine, or guanine.

The term “operably linked” is used to describe a DNA sequence and a regulatory sequence(s) connected in such a way as to permit gene expression when the appropriate molecules, e.g. transcriptional activator proteins, are bound to the regulatory sequence(s).

The terms “polypeptide” and “protein” are used interchangeably herein and includes chemically-modified polypeptides and/or polypeptides containing unnatural amino acid including D-amino acids. When appropriate, peptidomimetics that contain backbones other than the peptidyl bonds are contemplated.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

The term “recombinant” as used in reference to a nucleic acid indicates any nucleic acid that is positioned adjacent to one or more nucleic acid sequences that it is not found adjacent to in nature. A recombinant nucleic acid may be generated in vitro, for example by using the methods of molecular biology, or in vivo, for example by insertion of a nucleic acid at a novel chromosomal location by homologous or non-homologous recombination. The term “recombinant” as used in reference to a polypeptide indicates any polypeptide that is produced by expression and translation of a recombinant nucleic acid.

The term “transgene” is used herein to describe genetic material which has been or is about to be artificially inserted into the genome of an animal, particularly a mammalian cell of a living animal.

As used herein, the term “compound” includes, for example, pharmaceutical compounds, such as drugs and other biologically active compounds that may be administered in the treatment or prophylaxis of various medical indications or conditions. Such compounds are generally referred to herein as “therapeutic agents”. The term compound also includes pharmaceutical compounds that may be useful in diagnosis of various medical conditions or disorders. Disorders include diseases. Such compounds are generally referred to herein as “diagnostic agents.”

The term “corresponding” when used in reference to a gene means that a gene serves analogous, comparatively the same, functions in an organism to which it is endogenous compared to another gene endogenous to another organism. For example, a human hemoglobin gene corresponds to a bovine hemoglobin gene.

The terms “alteration”, “amino acid sequence alteration”, “variant” and “amino acid sequence variant” refer to noxin molecules with some differences in their amino acid sequences as compared to native noxin. Ordinarily, the variants will possess at least 70% homology with native noxin, and preferably, they will be at least about 80% homologous with native noxin. The amino acid sequence variants noxin falling within this invention possess a substitutions, deletions, and/or insertions at one or more positions. Certain variants retain substantial amount of activity of the unaltered, wild-type protein. Other variants lack such activity but compete with the wild type protein for binding or interaction, and thus act as inhibitors or other effectors without the wild-type activity.

The term “wild-type” means that the nucleic acid fragment or polypeptide does not comprise any mutations. A “wild-type” protein means that the protein will be active at a comparable level of activity found in nature and typically will comprise the amino acid sequence found in nature. A “wild-type” protein is usually most commonly found compared to “mutants,” and is part of normal cellular activities of a healthy cell. In an aspect of the invention, the term “wild type” or “parental sequence” can indicate a starting or reference sequence prior to a manipulation of the sequence.

### The Noxin Nucleic Acids

The present invention provides an isolated nucleic acid molecule comprising a polynucleotide encoding at least a portion of a polypeptide having an amino acid sequence selected from SEQ ID NOs: 2, 4, and 6. The nucleotide sequences for mouse (SEQ ID NO: 1; its deduced amino acid sequence is SEQ ID NO: 2), human (SEQ ID NO: 3; its deduced amino acid sequence is SEQ ID NO: 4), and rat (SEQ ID NO: 5, its deduced amino acid sequence is SEQ ID NO: 6) have been determined and disclosed herein.

Thus, one aspect of the invention provides an isolated polynucleotide comprising a nucleotide sequence selected from the group consisting of: (a) a nucleotide sequence encoding a polypeptide that has an amino acid sequence of SEQ ID NOs: 2, 4, and 6, corresponding polypeptides of other organisms, mutants or variants thereof, and any fragments thereof; (b) a nucleotide sequence depicted as SEQ ID NOs: 1, 3, and 5, corresponding genes from other organisms, allelic variants thereof, and fragments thereof; and (c) a nucleotide sequence complementary to at least one of any of the nucleotide sequences in (a) or (b) above. The embodiments of this aspect of the invention are further described below.

In certain embodiments of the invention, the isolated nucleic acid comprises a polynucleotide that encodes a noxin polypeptide, particularly a mouse noxin having the amino acid sequence SEQ ID NO: 2, human noxin having the amino acid sequence SEQ ID NO: 4, or a rat noxin having the amino acid sequence SEQ ID NO: 6. In other embodiments, the polynucleotide encodes a gene from other species corresponding to noxin.

In other embodiments, the polynucleotide of invention encodes a fragment of a noxin polypeptide. In particular embodiments, the fragment is biologically active and possesses some or all of biological activity of full length noxin protein. In particular embodiments, the polypeptide comprises at least 10, 12, 14, 16, 20, 22, 24, 25, 30, 35, 40, 50, 60, 70, 80, 90, 100, 110, 120, 130, 140, 150 contiguous amino acids depicted in SEQ ID NOs: 2, 4, and 6.

Some embodiments of the invention are isolated polynucleotides, each comprising a polynucleotide encoding a polypeptide, wherein the polypeptide has an amino acid sequence that is at least 80% but less than 100% identical, that is, 80%, 85%, 90%, 91%, 92%, 93%, 94%, 95%, 96%, 97%, 98%, 99%, 99.3, 99.6% or 100% identical to one of the sequences set forth in SEQ ID NOs: 2, 4, and 6 or any fragment thereof as described above.

Some embodiments of the invention that are isolated and/or recombinant nucleic acids having the nucleotide sequence depicted as SEQ ID NOs: 1, 3, and 5, and variants thereof. Variant nucleotide sequences include sequences that differ by one or more nucleotide substitutions, additions or deletions, such as allelic variants. Such variants may be naturally occurring or artificially introduced by molecular biological manipulation of the gene. In certain embodiments, polynucleotides encoding noxin polypeptide or fragments thereof, may be nucleic acids comprising a sequence that is at least 70% but less than 100% identical, that is 70%, 75%, 80%, 85%, 90%, 95%, 99%, 99.5%, or 99.7% identical to a sequence set forth in any one of SEQ ID NOs: 1, 3, and 5.

Methods of alignment of sequences for comparison are well known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. USA 8: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, (1988) Gene, 73: 237-244; Higgins and Sharp, (1989) CABIOS:11-13; Corpet, et al., (1988) Nucleic Acids Res., 16:881-90; Huang, et al., (1992) Computer Appl. Biosci. 8:1-7; and Pearson, et al., (1994) Methods Molec. Biol. 24:7-331. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Some of these polynucleotide variants are silent due to codon degeneracy, i.e. the polypeptides encoded by the variants have the amino acid sequence of SEQ ID NO: 2, 4, or 6. For example, a number of amino acids are designated by more than one triplet. Codons that specify the same amino acid, or synonyms (for example, CAU and CAC are synonyms for histidine) may result in “silent” mutations which do not affect the amino acid sequence of the protein. Others of these polynucleotide variants encode amino acid sequences that differ by one or more residues from SEQ ID NO: 2, 4, or 6, or are N-terminal fragments thereof due to a nonsense mutation. For uses involving expression of a noxin protein, nucleic acids that include changes that result in a nonsense codon within the coding region of noxin gene or having deletions or additions of one or two nucleotides that change the reading frame of the coding region are excluded from the variant nucleotide sequences contemplated herein. In other embodiments, variants are nucleotides with sequences that will hybridize under stringent hybridization conditions to a coding sequence of a nucleic acid sequence designated in SEQ ID NOs: 1, 3, and 5. By stringent hybridization condition it is meant that the calculated annealing temperature is Tm-25° C. See Howley et al. (1979) J. Biol. Chem. 254 (11): 4876. This is equivalent of hybridization temperature of 65° C. with 50% (w/w) formamide concentration.

In certain embodiments, nucleic acids of the invention comprise sequences identical to a fragment of any one of SEQ ID NOs: 1, 3, and 5. In one embodiment, such fragment is at least about 15, 20, 30, or 40 nucleotides in length which are useful as diagnostic probes and primers as discussed herein. Larger fragments 50-300 nucleotides in length are also useful according to the present invention, as are fragments corresponding to most, if not all, of at least one of the nucleotide sequences shown in SEQ ID NOs: 1, 3, and 5.

One skilled in the art will appreciate that these variations in one or more nucleotides of the nucleic acids encoding a particular protein may exist among individuals of a given species due to natural allelic variation. Any and all such nucleotide variations and resulting amino acid polymorphisms are within the scope of this invention.

Another aspect of the invention is a nucleic acid that is complementary to any of the nucleic acid described above. Such complementary nucleic acid may be DNA or RNA.

Polynucleotides having sequences encoding noxin (or their complement) have various applications in the art of molecular biology, including producing recombinant noxin proteins, uses as hybridization probes, in gene analysis and in the generation of anti-sense RNA and DNA. Such useful polynucleotide can code for a full-length noxin, or a fragment thereof.

In certain aspects, nucleic acids encoding noxin polypeptides or fragments thereof, and variants thereof may be used to increase expression of the gene in an organism or cell by direct delivery of the nucleic acid. Such introduction of noxin construct can be a nucleic acid therapy. The nucleic acid construct of the present invention can be delivered, for example, as an expression plasmid which, when transcribed in the cell, produces RNA which encodes a noxin polypeptide or fragments thereof, and genetically complement a partial or complete loss of function phenotype in the noxin gene. For example, a noxin nucleic acid of the invention may be expressed in a cell in which endogenous noxin has been knocked out or defective, and the introduced noxin nucleic acid will mitigate a phenotype resulting from the lack of endogenous noxin expression. In certain embodiments, using variant nucleic acid sequences, variant noxin polypeptide having more or less activities, or more or less stability can be introduced in a target cell.

In another aspect, nucleic acid encoding a noxin polypeptide, fragment thereof, or variants thereof, may be used to decrease gene expression. Such decrease can form a basis of a nucleic acid therapy. Useful construct can be delivered, for example, as an expression plasmid which, when transcribed in the cell, produces anti-sense RNA, which is complementary to at least a unique portion of the cellular mRNA which encodes a noxin polypeptide.

Alternatively, the construct is a single-stranded oligonucleotide which is generated ex vivo and which, when introduced into the cell, directly hybridizes with the mRNA and/or genomic sequences encoding the noxin polypeptide, thereby inhibiting noxin expression. Such oligonucleotide probes are optionally modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res. 48:2659-2668.

Another aspect of the invention relates to the use of RNA interference (RNAi) to effect knockdown of the noxin polypeptide gene described herein. “RNA interference” or “RNAi” is a term initially applied to a phenomenon observed in plants and worms where double-stranded RNA (dsRNA) blocks gene expression in a specific and post-transcriptional manner. RNAi is suitable to degrade specific mRNA sequences, and therefore inhibit expression of the target gene. RNAi constructs comprise RNA with a double-stranded portion that can specifically block expression of a target gene. The double-stranded portion may be formed by hairpin folding of a single RNA chain or by hybridization of two RNA chains. RNAi constructs can comprise either long stretches of double-stranded RNA identical or substantially identical to the target nucleic acid sequence or short stretches of double-stranded RNA identical or substantially identical to only a region of the target nucleic acid sequence. Exemplary methods of making and delivering either long or short RNAi constructs can be found, for example, in WO01/68836 and WO01/75164.

The noxin sequence disclosed herein is useful for identifying noxins and sequelogs in organisms other than mouse, human, or rat. For organisms with established complete genomic sequences, sequence homologies can be determined using the database of the genes within the genome. For other organisms without a complete genomic sequences, related sequences may be identified experimentally. The full-length native sequence gene encoding noxin, or portions thereof, may be used as hybridization probes for a cDNA library to isolate the full-length gene or to isolate still other genes (for instance, those encoding naturally-occurring variants of noxin from other species) which have a desired sequence identity to the noxin sequence disclosed in SEQ ID NOs: 1, 3, or 5, or from genomic sequences including promoters, enhancer elements and introns of native sequence noxin. For example, probes with length about 20 to about 50 bases are prepared. By way of example, a screening method will comprise isolating the coding region of the noxin gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the noxin gene of the present invention can be used to screen libraries of human cDNA, genomic DNA or mRNA to determine which members of such libraries the probe hybridizes to. Probes may also be employed in PCR techniques to generate a pool of sequences for identification of closely related noxin sequences.

Nucleotide sequences encoding the noxin polypeptide described herein can also be used to construct hybridization probes for the genetic analysis of individuals with genetic disorders. Under a stringent hybridization condition, a wild-type noxin probe or a mutant probe comprising one or more nucleotide variance is used to determine whether the genome of an individual is completely identical to any such probe. An individual with a variant noxin sequence may be at risk of or is afflicted by a disorder associated with stress, abnormal cell cycle or decreased cell death (i.e. cancer or other proliferative diseases such as psoriasis or rheumatoid arthritis).

### Noxin Proteins

In certain aspects, the invention provides isolated and purified noxin polypeptides or fragments thereof, of various animals, and functional variants thereof. In some embodiments, noxin polypeptides of the invention are isolated from, or otherwise substantially free of, other proteins which might normally be associated with the protein or a particular complex including the protein. In other embodiments, noxin peptides are associated with other functional peptides.

The invention provides polypeptides having signal peptides and polypeptides having them. Noxin polypeptide sequence contains nucleus localization signals, which may direct the peptides into nuclei. In one embodiment, the noxin polypeptide comprises the nuclear localization signal peptide. As signal peptides are normally cleaved from proteins post-translationally such that they are absent from the mature form of the protein, in another embodiment, noxin polypeptide lacks the signal peptide sequence. Accordingly, one embodiment of the invention provides the mature forms of the noxin polypeptides set forth in SEQ ID NO: 2, 4, and 6 lacking the localization signal, and variants thereof.

Variants of noxin polypeptides are also embodiments of the invention. The term “variants” encompasses polypeptides with amino acid substitutions, and also encompasses homologous genes of xenogeneic origin. In certain embodiments, the present invention includes the full-length variants of noxin proteins. In certain embodiments, a noxin polypeptide is a polypeptide comprising a portion of an amino acid sequence at least 80%, 85%, 90%, 91%, 92%, 93%, 94%, 95%, 96%, 97%, 98%, 99%, 99.3%, 99.6% or 100% identical to the amino acid sequence selected from SEQ ID NO: 2, 4, and 6, or to the mature forms of these noxin polypeptides. In other embodiments, the noxin polypeptide is a fragment of the full-length noxin, such as nucleic acids having peptide sequence that is partial sequence of SEQ ID NOs: 2, 4, and 6. In one embodiment, the fragments are N- or C-terminal fragments, preferably fragments having activities to cease apoptosis and/or to induce cell cycle arrest.

In certain embodiments, noxin variants, whether full-length or fragment, retain all, a substantial proportion, or at least partial biological activity. Some biological activities of noxin are the activities to cease apoptosis and/or to induce cell cycle arrest. By substantial activity, it is meant that the portion of the polypeptide retains, on a molar basis, at least 20%, 30%, 40%, 50%, 60%, 70%, 80%, 90% or 95% of the same activity of the mature, fully-active, wild-type protein. Such variants of a noxin polypeptide may have a hyperactive or constitutive activity.

In another aspect, the invention provides polypeptides that are antagonists of a noxin polypeptide and act to prevent the noxin polypeptide from performing one or more functions (“dominant negative effect”). In one embodiment, a truncated form of noxin, lacking one or more domains, has a dominant negative effect. In another embodiment, a mutant noxin polypeptide with one or more changes in the amino acid sequence has a dominant negative effect. Particularly, a mutant noxin polypeptide with changes in the phosphorylation sites so that it cannot be phosphorylated and does not mimic phosphorylated residue is considered for its dominant negative effect.

Optionally, a noxin polypeptide of the invention will function in place of an endogenous noxin polypeptide, for example, by mitigating a partial or complete loss of function phenotype in a cell. In an exemplary embodiment, a noxin polypeptide may be produced in a cell in which the endogenous noxin polypeptide has been reduced, and the introduced noxin polypeptide or receptor will mitigate a phenotype resulting from the reduction in endogenous expression.

Variants of noxin polypeptides may be useful for enhancing therapeutic or prophylactic efficacy, or stability (e.g., ex vivo shelf life and resistance to proteolytic degradation in vivo). In an embodiment, a variant has an intracellular half-life dramatically different than the corresponding wild-type protein. For example, the altered protein can be rendered either more stable or less stable to proteolytic degradation or other cellular process which result in destruction of, or otherwise inactivation of the noxin polypeptide of interest, thus allowing changing intracellular levels of noxin. A short half-life can give rise to more transient biological effects and, when part of an inducible expression system, can allow tighter control of recombinant noxin production within the cell.

Any of such modified polypeptides, with enhanced, differed, or reduced activities, can be produced, for instance, by amino acid substitution, deletion, or addition. For instance, it is reasonable to expect that an isolated replacement of a leucine with an isoleucine or valine, an aspartate with a glutamate, a threonine with a serine, cysteine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. Peptides with a mutation in the phosphorylation residue so that the residue is changed to glutamate or aspartate is considered for constitutional activation or nuclear localization in the absence of phosphorylation, and those with residue changed to alanine or other non-phosphorylatable residue is considered for nonactivatable or nuclear-localizable noxin.

Full-length and fragments of noxin polypeptides derived from a full-length noxin polypeptide may be prepared by several methods known to one skilled in the art. Isolated peptidyl portions of the subject proteins can be obtained by screening polypeptides recombinantly produced from the corresponding fragment of the nucleic acid encoding such polypeptides. The invention further provides noxin polypeptides, obtained when a nucleic acid comprising a nucleic acid sequence at least 85%, 90%, 95%, 97%, 98%, 99% or up to but not 100% identical to a nucleic acid sequence of SEQ ID NO: 1, 3, and 5 is expressed in cell. For uses involving expression of a noxin protein, nucleic acids that include changes that result in a nonsense codon within the coding region of noxin gene or having deletions or additions of one or two nucleotides that change the reading frame of the coding region are excluded from the variant nucleotide sequences contemplated herein. Ligating the polynucleotide sequence into a gene construct, such as an expression vector, and transforming or transfecting into hosts, either eukaryotic (yeast, avian, insect or mammalian) or prokaryotic (bacterial) cells, are standard procedures. In some embodiments, the cell is a bacterial cell or an insect cell. In a preferred embodiment, the cell is a mammalian cell. Vectors for such expression is described below in a Section named “Vectors.”

In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a noxin polypeptide or receptor.

This invention further contemplates a method of generating sets of combinatorial mutants of the noxin polypeptides, as well as truncation mutants, and is especially useful for identifying potential variant sequences (e.g. homologs). The purpose of screening such combinatorial libraries may be to generate noxin homologs, which can act as either agonists or antagonist, or alternatively, which possess novel activities all together. Combinatorially-derived homologs can be generated which have a selective potency relative to a naturally occurring noxin polypeptide. Such proteins, when expressed from recombinant DNA constructs, can be used in gene therapy protocols. The amino acid sequences for a population of noxin homologs are aligned, preferably to promote the highest homology possible. Such a population of variants can include, for example, homologs from one or more species, or homologs from the same species but which differ due to mutation. Amino acids which appear at each position of the aligned sequences may be selected to create a degenerate set of combinatorial sequences.

In a preferred embodiment, the combinatorial library is produced by way of a degenerate library of genes encoding a library of polypeptides which each include at least a portion of potential noxin sequences. For instance, a mixture of synthetic oligonucleotides can be enzymatically ligated into gene sequences such that the degenerate set of potential noxin nucleotide sequences are expressible as individual polypeptides, or alternatively, as a set of larger fusion proteins (e.g. for phage display).

There are many ways by which the library of potential homologs can be generated from a degenerate oligonucleotide sequence. The synthesis of degenerate oligonucleotides is well known in the art (see for example, Narang, S A (1983) Tetrahedron Let. 39:3; Itakura et al., Recombinant DNA, Proc. 3rd Cleveland Sympos. Macromolecules, ed. AG Walton, Amsterdam: Elsevier pp 273-289 (1981); Itakura et al., (1984) Annu. Rev. Biochem. 53:323; Itakura et al., (1984) Science 198:1056; Ike et al., (1983) Nucleic Acid Res. 11:477). Chemical synthesis of a degenerate gene sequence can be carried out in an automatic DNA synthesizer, and the synthetic genes then be ligated into an appropriate gene for expression.

Alternatively, other forms of mutagenesis can be utilized to generate a combinatorial library. For example, noxin variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell. Biol. 12:2644-2652; McKnight et al., (1982) Science 232:316); by saturation mutagenesis (Meyers et al., (1986) Science 232:613); by PCR mutagenesis (Leung et al., (1989) Method Cell. Mol. Biol. 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies Mol. Biol. 7:32-34). Linker scanning mutagenesis, particularly in a combinatorial setting, is an attractive method for identifying truncated (bioactive) forms of noxin polypeptides.

Covalent modifications of noxin are included within the scope of this invention. One type of covalent modification includes reacting targeted amino acid residues of the noxin polypeptide with an organic derivatizing agent that is capable of reacting with selected side chains or the N- or C-terminal residues of noxin. Derivatization with bi-functional agents is useful, for instance, for crosslinking noxin to a water-insoluble support matrix or surface for use in the method for purifying anti-noxin antibodies, and vice-versa. Commonly used crosslinking agents include e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

In other embodiments, the invention includes fusion proteins including at least a portion of the noxin polypeptide and a portion of another polypeptide, which serves as a tag sequence for detection or purification handle.

Various tag polypeptides and their respective antibodies are well known in the art. In the instant invention, the FLAG-peptide (Hopp et al., (1988) BioTechnology, 6:1204-1210) was used but others are available and within the skill of one of the relevant art. Examples include poly-histidine (poly-his) or poly-histidine-glycine (poly-his-gly) tags; the influenza hemagglutinin (HA) tag polypeptide and its antibody 12CA5 (Field et al., (1988) Mol. Cell. Biol., 8:2159-2165); the c-myc tag and the 8F9, 3C7, 6E10, G4, B7 and 9E10 antibodies thereto (Evan et al., (1985) Mol. Cell. Biol., 5:3610-3616); and the Herpes simplex virus glycoprotein D (gD) tag and its antibody (Paborsky et al., (1990) Protein Engineering, 3(6):547-553). Other tag polypeptides include the KT3 epitope peptide (Martin et al., (1992) Science, 255:192-194); an α-tubulin epitope peptide (Skinner et al., (1991) J. Biol. Chem., 266:15163-15166); and the T7 gene 10 protein peptide tag (Lutz-Freyermuth et al., (1990) Proc. Natl. Acad. Sci. USA, 87:6393-6397). In a certain embodiment, the fusion protein is a GST fusion protein, intein fusion protein, or cellulose binding domain fusion protein. These fusion/tag proteins facilitate detection and purification.

Techniques for making these fusion genes are well known. Essentially, the joining of various DNA fragments coding for different polypeptide sequences is performed in accordance with conventional molecular biological techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation, all while mindful of desired coding frame. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992, and its updates).

### Antibodies

Another aspect of the invention pertains to an antibody reactive with an noxin polypeptide, preferably antibodies that are specifically reactive with said proteins. For example, by using immunogens derived from a noxin polypeptide, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster, or rabbit can be immunized with an immunogenic form of the peptide (e.g., a noxin polypeptide, or an antigenic fragment which is capable of eliciting an antibody response, or a fusion protein). Techniques for conferring immunogenicity on a protein or peptide include conjugation to carriers or other techniques well known in the art. An immunogenic portion of a noxin polypeptide can be administered, alone or conjugated to a carrier such as keyhole limpet hemocyanin, in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a noxin polypeptide of a mammal, e.g., antigenic determinants of a protein set forth in SEQ ID NOs: 2, 4, and 6. In one embodiment, antibodies are specific for a noxin protein having the amino acid sequence as set forth in any of SEQ ID NOs: 1, 3, and 5.

Following immunization of an animal with an antigenic preparation of a noxin polypeptide, anti-noxin antisera can be obtained and, if desired, polyclonal anti-noxin antibodies can be isolated from the serum.

In certain embodiments, an antibody of the invention is a monoclonal antibody. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Once obtained, a hybridoma can be propagated in a cell culture, optionally in culture conditions where the hybridoma-derived cells produce the monoclonal antibody that binds specifically to the noxin polypeptide or fragments thereof. The monoclonal antibody may be purified from the cell culture.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g. by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays, and immunohistochemistry.

Another application of anti-noxin antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of a noxin polypeptide, e.g., other orthologs of noxin or other paralogs and/or sequelogs from the same species, can then be detected with antibodies, as, for example, reacting nitrocellulose filters lifted from infected plates with the appropriate anti-noxin antibodies. Positive phage detected by this assay can then be isolated from the infected plate. Thus, the presence of noxin homologs can be detected and cloned from other animals, as can alternate isoforms (including splice variants) from humans.

The antibodies described herein may be used to assay the levels of the noxin polypeptides described herein, and in particular for detecting the presence of a noxin polypeptide on a biological sample. The level of noxin polypeptide may be measured in a variety of sample types such as, for example, in cells, stools, and/or in bodily fluid, such as in whole blood samples, blood serum, blood plasma, urine, and tissue samples. An antibody specifically reactive with noxin is preferable. The adjective “specifically reactive with” as used in reference to an antibody is intended to mean, as is generally understood in the art, that the antibody is sufficiently selective between the antigen of interest (e.g. a noxin polypeptide) and other antigens that are not of interest that the antibody is useful for, at minimum, detecting the presence of the antigen of interest in a particular type of biological sample.

In certain methods employing the antibody, a higher degree of specificity in binding may be desirable. For example, an antibody for use in detecting a low abundance protein of interest in the presence of one or more very high abundance protein that are not of interest may perform better if it has a higher degree of selectivity between the antigen of interest and other cross-reactants. Monoclonal antibodies generally have a greater tendency (as compared to polyclonal antibodies) to discriminate effectively between the desired antigens and cross-reacting polypeptides. In addition, an antibody that is effective at selectively identifying an antigen of interest in one type of biological sample (e.g. a stool sample) may not be as effective for selectively identifying the same antigen in a different type of biological sample (e.g. a blood sample). Likewise, an antibody that is effective at identifying an antigen of interest in a purified protein preparation that is devoid of other biological contaminants may not be as effective at identifying an antigen of interest in a crude biological sample, such as a blood or urine sample. Accordingly, in preferred embodiments, the method employs antibodies that have demonstrated specificity for an antigen of interest in a sample type that is likely to be the sample type of choice for use of the antibody. In a particularly preferred embodiment, the method uses antibodies that bind specifically to a noxin polypeptide in a protein preparation from frozen tissue samples (optionally testis, heart, spleen, brain, lung, skeletal muscle, kidney or liver) from a subject.

### Vectors

In another aspect of the invention, the subject nucleic acid is provided in an expression vector comprising a noxin nucleotide sequence (e.g. SEQ ID NO: 1, 3, or 5) encoding a subject noxin polypeptide (e.g. SEQ ID NOs: 2, 4, or 6) or a fragment thereof, operably linked to at least one regulatory sequence suitable for expression of the polypeptide in either prokaryotic cells, eukaryotic cells, or both. Regulatory sequences are art-recognized and are selected to direct expression of the polypeptide, or a fragment. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). Expression vehicles for production of a recombinant noxin polypeptide include plasmids and other vectors. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

For instance, suitable vectors for the expression of a polypeptide include plasmids of the types: pBR322-derived plasmids, pEMBL-derived plasmids, pEX-derived plasmids, pBTac-derived plasmids, and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo, pHyg, pENTR-TOPO-D and pMSCV-puro derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17.

Other vectors useful for expression of noxin peptides include baculovirus based vectors (See, for example, U.S. Pat. No. 4,745,051; exemplary vectors: pVL1393, pAcAB3; kits commercially available include ProEasy™ sold by AB Vector, San Diego, Calif., and BaculoDirect™ Baculovirus Expression System sold by Invitrogen Corp., Carlsbad, Calif.). These Autographa california nuclear polyhedrosis virus-based vectors can be used by transfecting insect cells such as Sf9 (Spodoptera frugiperda) or High Five™ (Trichospora ni) cells.

In certain embodiments, the vector is created to encode one of six possible noxin shRNAs, under the control of the human U6 promoter.

### Method of Producing Polypeptides

The present invention further pertains to methods of producing the subject noxin polypeptides. A nucleotide sequence encoding a noxin polypeptide can be used to produce a recombinant form of the protein via microbial or eukaryotic cellular processes. For example, a host cell (further described below) transfected with an expression vector encoding a noxin polypeptide as described above, can be cultured under appropriate conditions to allow expression of the polypeptide to occur. With an appropriate signal sequence, the polypeptide may be secreted and isolated from a mixture of cells and medium containing the polypeptide. Alternatively, the polypeptide may be retained cytoplasmically or in a membrane fraction and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide.

It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) Proc. Nat. Acad. Sci. USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a noxin polypeptide. For example, the VP6 capsid protein of rotavirus can be used as an immunologic carrier protein for portions of polypeptide, either in the monomeric form or in the form of a viral particle. Another example is a nucleic acid sequence corresponding to the portion of the noxin polypeptide incorporated into a fusion gene construct which includes coding sequences for a late vaccinia virus structural protein, to produce a recombinant virus expressing fusion protein comprising a portion of the protein as part of the virion. The Hepatitis B surface antigen can also be utilized in this role as well. Similarly, chimeric constructs coding for fusion proteins containing a portion of a noxin polypeptide and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni⁺² metal resin. The purification leader sequence can then be subsequently removed by treatment with enterokinase to provide the purified interleukin/interferon polypeptide or receptor (e.g., see Hochuli et al., (1987) J. Chromatography 411:177; and Janknecht et al., (1991) Proc. Nat. Acad. Sci. USA 88:8972).

Forms of noxin may be recovered from culture medium or from host cell lysates. It may be desired to purify the noxin from recombinant cell proteins or polypeptides. The following procedures are exemplary of suitable purification procedures: by fractionation on an ion-exchange column; ethanol precipitation, reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the interleukin/interferon. Various methods of protein purification may be employed and such methods are known in the art and described for example in Deutscher, Methods in Enzymology, 182 (1990); Scopes, Protein Purification: Principles and Practice, Springer-Verlag, New York (1982).

### Transgenic Animals

Another aspect of the invention features transgenic, non-human animals or progeny or embryo thereof, which express a heterologous noxin gene. In another aspect the invention features transgenic non-human animals which have had one or both copies of the endogenous noxin genes disrupted in at least one of the tissue or cell-types of the animal. In one embodiment, the transgenic non-human animals is a mammal such as a mouse, rat, rabbit, goat, sheep, dog, cat, cow, or non-human primate. In one embodiment, the noxin transgenic animals of the present invention may be used for in vivo assays to identify anti-cancer or anti-viral therapeutics.

Transgenic animals are created by introducing an exogenous gene into such animals. Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

DNA constructs for random integration need not include regions of homology to mediate recombination. Where homologous recombination is desired, the DNA constructs will comprise at least a portion of the target gene with the desired genetic modification, and will include regions of homology to the target locus. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

When ES cells have been transformed, they may be used to produce transgenic animals. An ES cell line may be employed, or embryonic cells may be obtained freshly from a host, e.g. mouse, rat, guinea pig, rabbit, etc. Such cells are grown on an appropriate fibroblast-feeder layer or grown in the presence of appropriate growth factors, such as leukemia inhibiting factor (LIF). After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture.

The transgenic animals described herein may comprise a wild-type gene, naturally occurring polymorphic gene, xenogeneic (i.e. from a different species than the animal host) gene, or a gene that contains alterations to endogenous genes in addition to, or alternatively, to the genetic alterations described above. The introduced gene may have a genetically manipulated sequence, for example having deletions, substitutions, or insertions in the coding or non-coding regions. For example, the host animals may be either “knockouts” and/or “knockins” for a target gene(s) as is consistent with the goals of the invention (e.g., the host animal's endogenous noxin may be “knocked out”). Knockouts have a partial or complete loss of function in one or both alleles of an endogenous gene of interest. Knockins have an introduced transgene with altered genetic sequence and/or function from the endogenous gene. The two may be combined, for example, such that the naturally occurring gene is disabled, and an altered form introduced. For example, it may be desirable to knockout the host animal's endogenous noxin gene, while introducing an exogenous noxin gene (e.g., a human noxin gene).

A “knockin” of a target gene means an alteration in a host cell genome that results in altered expression or function of a native target gene. Increased (including ectopic) or decreased expression may be achieved by introduction of an additional copy of the target gene, or by operatively inserting a regulatory sequence that provides for enhanced expression of an endogenous copy of the target gene. These changes may be constitutive or conditional, i.e. dependent on the presence of an activator or repressor. The use of knockin technology may be combined with production of exogenous sequences to produce the transgenic animals of the invention.

In one aspect of the invention, a noxin transgene can encode the wild-type form of the protein, homologs thereof, as well as antisense constructs. A noxin transgene can also encode a soluble form of the protein that has immunomodulatory and/or antiproliferative activity. It may be desirable to express the heterologous noxin transgene conditionally such that either the timing or the level of noxin gene expression can be regulated. Transgenic animals containing an inducible noxin transgene can be generated using inducible regulatory elements (e.g., metallothionein promoter), which are well-known in the art. Noxin transgene expression can then be initiated in these animals by administering to the animal a compound which induces gene expression (e.g., heavy metals). Another preferred inducible system comprises a tetracycline-inducible transcriptional activator (U.S. Pat. Nos. 5,654,168 and 5,650,298). Another conditional expression can be provided using prokaryotic promoter sequences, which require prokaryotic proteins to be simultaneous expressed in order to facilitate expression of the noxin transgene. Exemplary promoters and the corresponding trans-activating prokaryotic proteins are given in U.S. Pat. No. 4,833,080.

Moreover, transgenic animals exhibiting tissue specific expression can be generated, for example, by inserting a tissue specific regulatory element, such as an enhancer, into the transgene. For example, the endogenous noxin gene promoter or a portion thereof can be replaced with another promoter and/or enhancer, e.g., a CMV or a Moloney murine leukemia virus (MLV) promoter and/or enhancer.

The present invention provides also for transgenic animals that carry the transgene in all their cells, as well as animals that carry the transgene in some, but not all cells, i.e., mosaic animals. The transgene can be integrated as a single transgene or in tandem, e.g., head to head tandems, or head to tail or tail to tail or as multiple copies.

The successful expression of the transgene can be detected by any of several means well known to those skilled in the art. Non-limiting examples include Northern blot, in situ hybridization of mRNA analysis, Western blot analysis, immunohistochemistry, and FACS analysis of protein expression.

In one embodiment of the invention, the non-human transgenic mammal or progeny or embryo thereof has integrated into its genome DNA a disrupted noxin gene. In homozygous individuals of such transgenic animals, no noxin protein is produced.

In such a “knockout,” preferably the target gene expression is undetectable or insignificant. For example, a knock-out of a noxin gene means that function of the noxin has been substantially decreased so that expression is not detectable or only present at insignificant levels. This may be achieved by a variety of mechanisms, including introduction of a disruption of the coding sequence, e.g., insertion of one or more stop codons, insertion of a DNA fragment, deletion of coding sequence, substitution of stop codons for coding sequence, etc. In some cases the exogenous transgene sequences are ultimately deleted from the genome, leaving a net change to the native sequence. Different approaches may be used to achieve the “knock-out”. A chromosomal deletion of all or part of the native gene may be induced, including deletions of the non-coding regions, particularly the promoter region, 3′ regulatory sequences, enhancers, or deletions of gene that activate expression of APP genes. A functional knock-out may also be achieved by the introduction of an anti-sense construct that blocks expression of the native genes (for example, see Li and Cohen (1996) Cell 85:319-329). “Knock-outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration post-natally.

In certain embodiments, the invention further provides methods for identifying (screening) or for determining the safety and/or efficacy of therapeutics, i.e. compounds which are useful for mitigating stress-induced cell damage or for inducing or preventing cell death. In addition, the assays are useful for further improving known therapeutic compounds, e.g., by modifying their structure to increase their stability and/or activity and/or toxicity. In an example, animals are subjected to defined stress conditions such as physical or chemical injury, infection, inflammatory conditions, or oxidative stress condition. A candidate agent is administered to transgenic mice lacking noxin before, during, or after stress conditions are imposed, and the effect of the stress on organs and tissues such as liver, heart, blood vessels, lung, kidney, pancreas, lymph, is determined by comparing the test animals with control animals. Because mice lacking noxin are more sensitive to stress, it is expected that a beneficial candidate would more clearly be discernable as compared to when such test is carried out using wild-type mice. In another example, transgenic animals received solid tumor transplants. A candidate agent is administered and the effect on the tumor growth is determined.

### Cells

This invention also pertains to a host cell transfected with a recombinant gene, including a coding sequence for one or more of the subject noxin polypeptides. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

In a further aspect, the invention features non-human animal cells containing a noxin transgene. For example, the animal cell (e.g. somatic cell or germ cell (i.e. egg or sperm)) can be obtained from the transgenic animal. Transgenic somatic cells or cell lines can be used, for example, in drug screening assays. Transgenic germ cells, on the other hand, can be used in generating transgenic progeny, as described below.

Transgenic cells can also be used for in vitro screening for agents that protect cells from stress, induce cell cycle arrest, or enhance cell death, as further described below.

### Applications

One aspect of the invention provides methods of protecting a cell from stress damage by enhancing the expression of noxin and/or enhancing noxin activity. As disclosed in the instant specification and concretely demonstrated in the Examples below, noxin is expected to protect cells from stress induced damages. In one embodiment, noxin activity is increased by enhancing the mRNA expression of noxin, thus increasing the noxin polypeptide. In particular, noxin mRNA expression is enhanced by introducing exogenous noxin gene in an expression vector suitable for the target cells. In another embodiment, noxin activity is increased by activating noxin polypeptide. Further, noxin expression is dependent on p53. As such, in one embodiment, noxin expression is enhanced by enhancing p53 expression, or by preventing inhibition of p53.

Noxin polypeptide is phosphorylated in response to NO inducing stress factors. The phosphorylation sites fit the concensus for DNA-PK, Akt, and/or ATM. Thus, in one embodiment, noxin activity is controlled by administering an agonist or antagonist of DNA-PK, Akt, and/or ATM. In certain embodiments, the stress damage is caused by γ-irradiation, UV-irradiation, adriamycin, activated oxygen such as hydrogen peroxide, cytokines, or nitrogen-donors, e.g., S-nitroso-N-acetyl-D,L-penicillamine (SNAP), 1-hydroxy-2-oxo-3-(N-ethyl-2-aminoethyl)-3-ethyl-1-triazene (NOC12), and DETA NONOate (NOC18).

Another aspect of the invention is a method of preventing or decreasing cell death caused by stress by increasing noxin activity in the cell. In one embodiment, noxin activity is increased by increasing noxin mRNA expression. In another embodiment, noxin activity is increased by promoting noxin polypeptide translation. In another embodiment, noxin activity is increased by enhancing noxin activation of expressed noxin polypeptide. Noxin expression or activities are regulated as described above. The stress damage may be caused by any of the stress factors described above.

In yet another aspect, the invention is a method of inducing cell death by inhibiting noxin activity. In one embodiment, noxin activity is inhibited by inhibiting noxin mRNA expression. In another embodiment, noxin activity is inhibited by inhibiting noxin polypeptide translation. Noxin expression is inhibited by RNAi technology, as described in the Examples. Briefly, short hairpin RNA with specific sequences are introduced into cells, processed by cellular machinery into single strands, and hybridize with noxin mRNA, forming an RNA complex which is then specifically degraded. In another embodiment, noxin activity is inhibited by inhibiting noxin activation of expressed noxin polypeptide. Phosphorylation of noxin, which appears to be important in translocation and activation of noxin proteins, is inhibited by kinase inhibitors inhibiting the activity of DNA-PK, Akt, and/or ATM. In certain embodiment, noxin activity is inhibited by inhibiting p53 activity.

Another aspect of the invention is a method of inducing cell cycle arrest by increasing noxin activity. In one embodiment, noxin activity is increased by increasing noxin mRNA expression. In another embodiment, noxin activity is increased by promoting noxin polypeptide translation. In another embodiment, noxin activity is increased by enhancing noxin activation of expressed noxin polypeptide. The noxin expression or activity is regulated as above, except that p53 has no effect on cell cycle regulation by noxin and therefore a method involving p53 is not contemplated.

Another aspect of the invention is a method of preventing cell cycle arrest by decreasing noxin activity. In one embodiment, noxin activity is inhibited by inhibiting noxin mRNA expression. In another embodiment, noxin activity is inhibited by inhibiting noxin polypeptide translation. In another embodiment, noxin activity is inhibited by inhibiting noxin activation of expressed noxin polypeptide. The noxin expression or activity is regulated as above, except that p53 has no effect on cell cycle regulation by noxin and therefore a method involving p53 is not contemplated.

Another aspect of the invention is a method of evaluating damage of the genome in a cell by detecting levels of noxin expression in the cell. In one embodiment, the expression level is noxin mRNA quantity. In another embodiment, the expression level is noxin polypeptide quantity.

An aspect of the invention is methods to identify an agent that affects noxin expression and/or activity. Once a system is validated using a known inhibitor or enhancer of noxin expression and/or activity, candidate agents are tested using transgenic cells expressing exogenous noxin to identify agents that decrease the expression and/or activity of noxin. In another aspect, candidate agents are tested using transgenic cells expressing reduced or undetectable amount of noxin. Agents that mimic noxin by stimulating downstream enzymes, and therefore restoring wild-type phenotypes, may be useful in treating conditions where noxin is missing or defective.

A different aspect of the invention comprises methods of identifying status of cell with regard to apoptosis by measuring noxin mRNA and/or protein and/or activities. Cells that are heading into apoptosis are expected to contain decreased amount of noxin compared to wild-type cells. Noxin can be detected by northern blot, Q-PCR, western blot, or flow cytometry.

Noxin is also part of the normal development of heart, testis, spleen, or bone marrow. Certain embodiments of the invention are methods of protecting heart, testis, spleen, or bone marrow against stress factors, by administering to an animal suffering from, suffered from, or expected to suffer from (for example, by surgery) stress a therapeutically effective amount of noxin. In other embodiments, noxin is administered to repair damaged or underdeveloped heart, testis, spleen, or bone marrow. The method of administration would be known to one in the art and the adjustment of dosage is routine for one skilled in the art. In another embodiment, endogenous noxin is induced by controlled administration of stress factors such as H₂O₂.

### EXEMPLIFICATION

### Example 1

### Identification of the Noxin Gene

NIH3T3 cells were maintained at 37° C. in an atmosphere of 5% CO₂ in Dulbecco modified Eagle's medium (DMEM) with 10% calf serum, and were treated with three different donors of nitric oxide (NO), S-nitroso-N-acetyl-D,L-penicillamine (SNAP), 1-hydroxy-2-oxo-3-(N-ethyl-2-aminoethyl)-3-ethyl-1-triazene (NOC12), and DETA NONOate (NOC18), at the concentration of 250 μM. Total RNA was isolated from cells as follows: Cells cultured in 10 cm plates were lysed with 2 ml of TRIzol (invitrogen) and poly(A)⁺ RNA was selected with FastTrack 2.0 (Invitrogen) according to the manufacturer's protocol. The isolated mRNA was resuspended in TE (10 mM Tri-HCl and 1 mM EDTA, pH 7.4) buffer at the final concentration of 2 μg/μl. and NO-treated cells and polyA⁺ RNA isolated.

NO-induced gene expression changes were determined by cDNA microarray analysis as described by Hemish et al., (2003) J. Biol. Chem. 278:42321-9. Briefly, cDNA probes prepared from polyA⁺ RNA isolated from untreated and NO-induced cells were labeled with Cy5 or Cy3 fluorescent dyes and hybridized to custom printed slide arrays produced by the Cold Spring Harbor Laboratory Genome Center. Each slide was comprised of 9,600 features, encompassing both expressed sequence tags (˜47%) and known genes (˜53%) (acquired from the NIA, National Institutes of Health, Bethesda, Md.; http://Igsun.grc.nia.nih.gov/cDNA/cDNA.html), along with appropriate controls, such as housekeeping genes and marker genes to monitor by us predicted gene expression profiles. cDNAs of NO-inducible genes identified by us previously were incorporated into the array. For hybridization, the slides were hydrated by exposure to 1× saline sodium citrate for 1.5 min at 30° C. in a humid chamber, snap-dried on a 100° C. heating plate, rinsed in 0.1% SDS for 30 s, rinsed in distilled water for 30 s, boiled in distilled water for 5 min, then rinsed in −20° C. benzene-free ethanol, and spun dry at 2,000×g for 5 min. cDNA samples were resuspended in buffer (0.3 M sodium bicarbonate, pH 9.0), coupled to monofunctional NHS-Cy5 or Cy3 dye (Amersham Biosciences) for 15 min at 25° C., purified using Microcon 30 columns, and combined with mouse Cot-1 DNA (20 μg), poly(A)⁺ RNA (2 μg), and tRNA (2 μg). The microarrays were hybridized using GlassHyb hybridization solution (Clontech Laboratories) and washed according to the manufacturer's instructions. Hybridizations for the complete time course were performed in triplicate with color reversals for each individual time point, resulting in a total of six replicates/time point. To minimize variability, all of the samples from each experimental treatment were simultaneously hybridized, washed, and scanned. After washing, the microarray slides were scanned using GenePix scanner and software (version 3.0; Axon Instruments, Inc.). Fluorescence intensities for all spots were exported to the data analysis software, GeneSpring 4.2 (Silicon Genetics, Redwood City, Calif.) and normalized by the “per chip normalization” method. Expression ratio values obtained from the six independent replicates were averaged for each experimental time point and filtered for changes that were statistically significant (p<0.05, compared with reference by Student's t test for each time point) and either up-regulated or down-regulated 1.3-fold. Expression profiles of the filtered data set were further analyzed for coordinated expression patterns and functional information by using the hierarchical clustering program in the GeneSpring program suite. Functional annotation was performed by searching NCBI Protein Database and SOURCE database (http://source.stanford.edu).

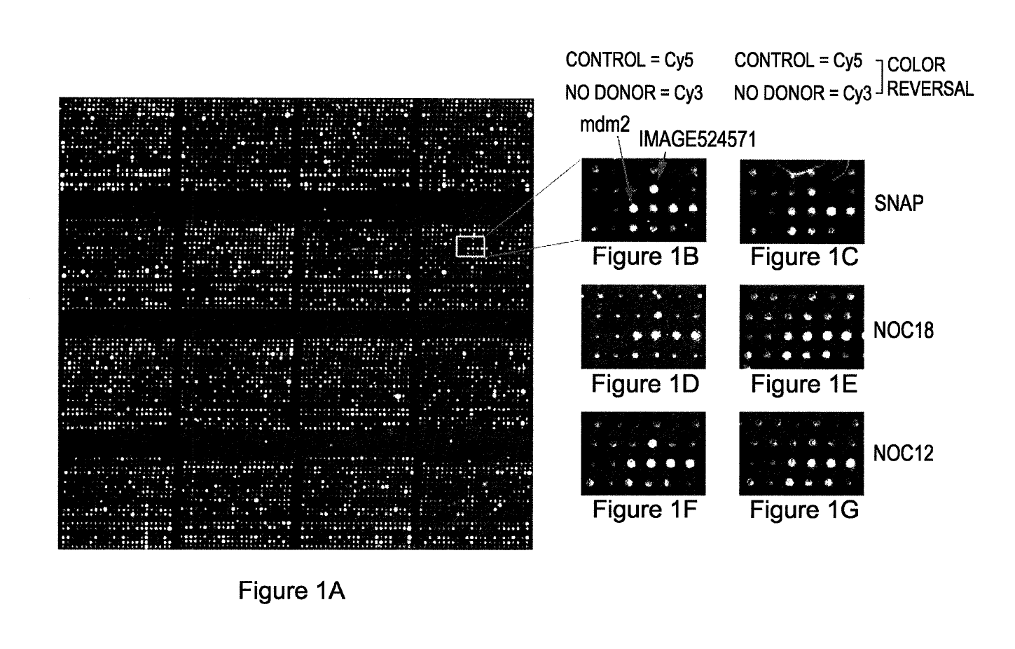

FIG. 1A is a panel of microarray images showing noxin induction by NO donor SNAP. Panels 1B-G show magnified view of a section of the microarray showing positive signals from RNA samples treated with SNAP (panels 1B and 1C), NO12 (panels 1D and 1E), and NO18 (panels 1F and 1G). Panels 1B, 1D, and 1F show control samples labeled with Cy5 and NO-induced samples labeled with Cy3, and Panels 1C, 1E, and 1G show the same experiment with color reversed by labeling control samples with Cy3 and NO-induced samples labeled with Cy5.

Up-regulation of two distinct genes can be seen in a section of the array: (1) one EST gene, IMAGE524571 (noxin), the function of which was previously unidentified and (2) mdm2 (murine double minute oncogene), a negative regulator of p53 tumor suppression, were strongly induced by all three NO donors. Mdm2 is known in the art as an example of a gene induced strongly by NO.

### Example 2

### Molecular Cloning of the Noxin Gene

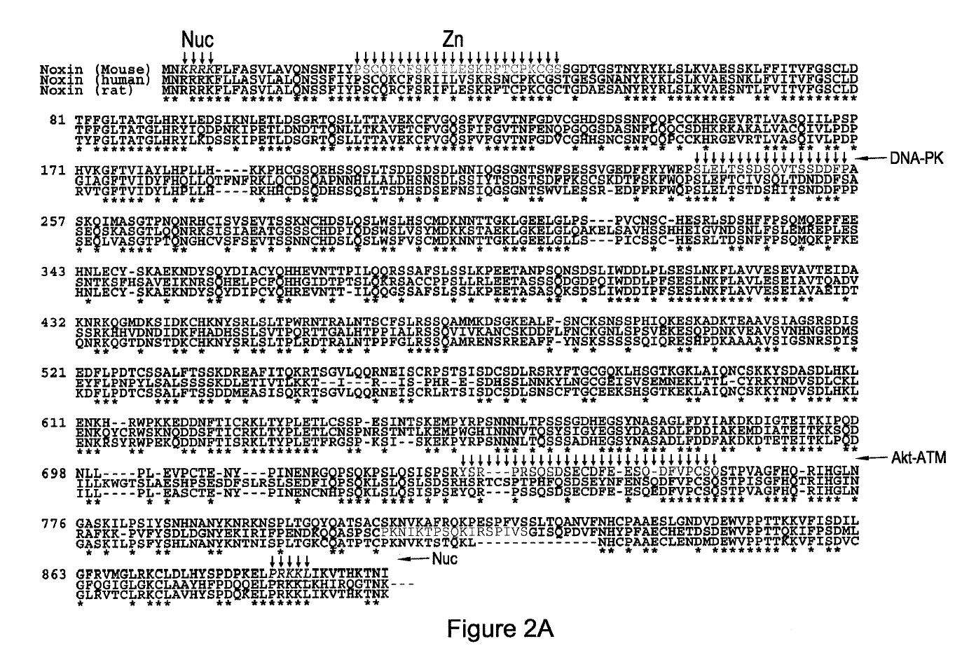

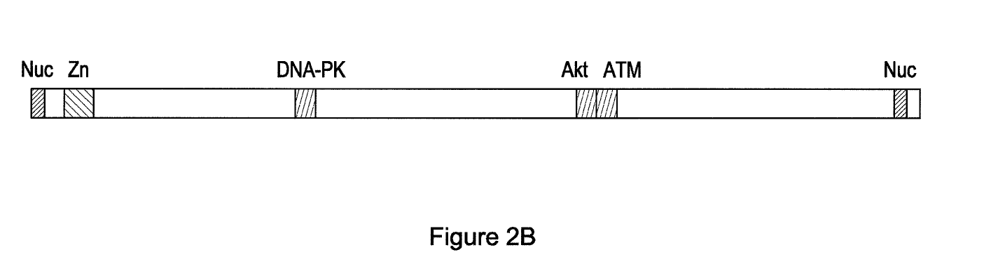

IMAGE524571 sequence from example 1 was used for further searches. A 3384-bp cDNA clone (GeneID:74041), which contained the entire coding region of the gene, was identified in the NCBI database. 5′-RACE analysis described below confirmed that this cDNA sequence contains the 5′-end of the mRNA (with a stop codon preceding the open reading frame) and codes for a protein of 898 amino acids (SEQ ID NO: 2, FIG. 2A first line), with a predicted molecular weight of 100 kD and an isoelectric point of 7.23 (sequence submitted as DQ400346 to GENBANK).

The 5′-RACE analysis technique performed consisted of using the FirstChoice RLM-RACE kit (Ambion) according to manufacturer's instructions. PolyA⁺ RNA isolated from adult mouse testes was reverse transcribed into cDNA. A specially designed adaptor sequence provided in the RLM-RACE kit was ligated to the ends of the cDNA, and the adaptor primer served as the forward primer. An antisense gene-specific primer (5′-GAAGCACCTTTGACATGATGGA-3′ (SEQ ID NO:11)) derived from nucleotides 270-291 in the noxin cDNA, and antisense gene-specific primer (5′-TGGCCCTTCTTGGCATAATG-3′ (SEQ ID NO:12)) derived from nucleotides 125-144, served as the outer and nested primers, respectively. PCR products were cloned into pCRII-TOPO vector (Invitrogen) and sequenced.

Cloned mouse cDNA of 3,384 bp was sequenced (SEQ ID NO: 1) using standard laboratory techniques and the 898 amino acid-coding region was deduced (SEQ ID NO: 2). Corresponding human (SEQ ID NO: 3) and rat (SEQ ID NO: 5) nucleotide sequences and partial protein sequences (human, SEQ ID NO: 4; rat, SEQ ID NO: 6) were generated using EST and genome databases. The protein sequences are depicted in FIG. 2A. Asterisks denote identical amino acids for all three species.

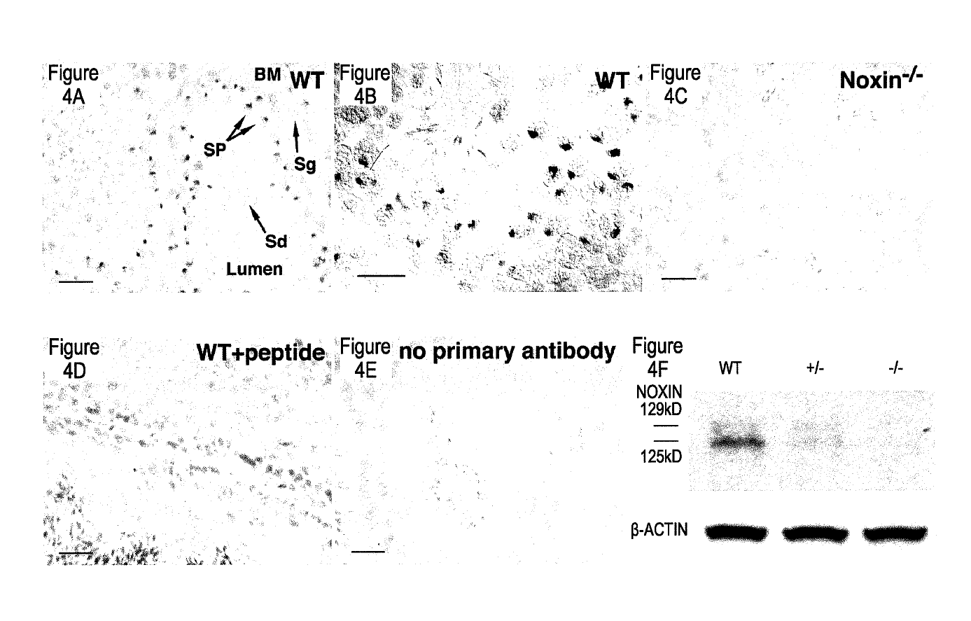

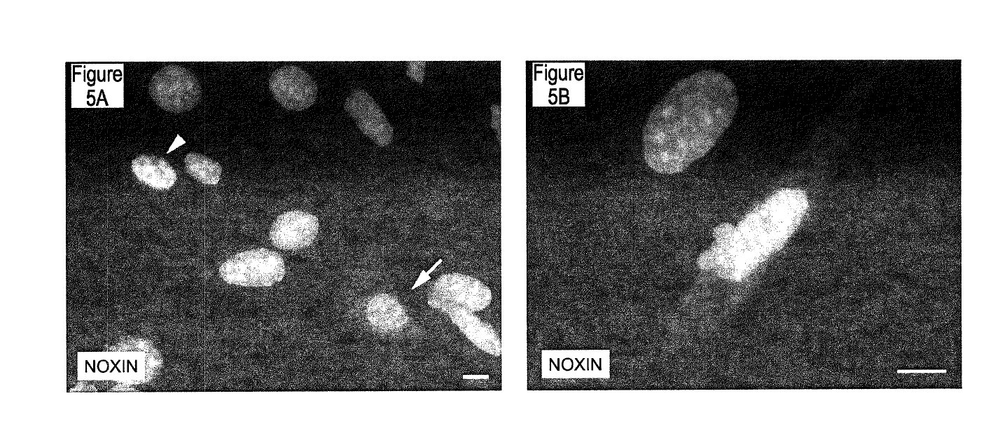

As shown in FIG. 2A, analysis of the polypeptide sequence suggests noxin contains (1) a C3HC4 type zinc-finger-like domain (light blue) in the N-terminal region, (2) DNA-PK, ATM, and Akt phosphorylation consensus sequences (pink), and (3) two potential nuclear localizing signals (red, italics). The light purple text represents an overlap of the ATM and Akt consensus sequences. The deduced noxin protein sequence is highly rich in serines (134 serine residues), an unusual feature suggesting phosphorylation of these residues as a possible modification. As evidence supporting this functional characteristic, in the extracts of both the animal tissue and cultured NIH3T3 cells after transfection with the noxin cDNA, two distinct bands were apparent on the SDS-polyacrylamide gel (see FIGS. 4F, 6C, 8, and 9) and the intensity of the upper band was markedly reduced upon phosphatase treatment (data not shown). Potential phosphorylation sites, as well as elements identified above, are shown in a schematic (FIG. 2B).

### Example 3

### Location of the Noxin Gene in the Genome

Based on the mouse genome map, the noxin gene is located on mouse chromosome 7. The gene is conserved across the species (70% similarity at the DNA level) and can be found in the rat and human genomes (located on rat chromosome 1 and human chromosome 11). There appears to be a single copy of noxin gene in each of the genome of these species.

### Example 4

### Cloned Noxin cDNA Generates a Polypeptide with an Endogenous Counterpart

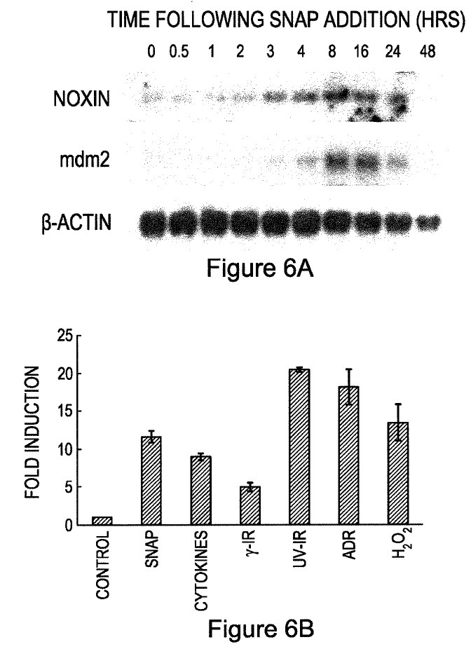

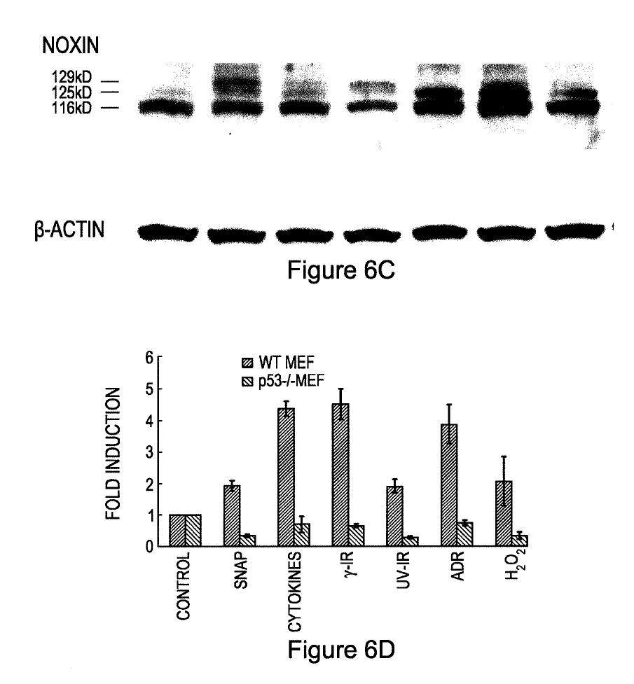

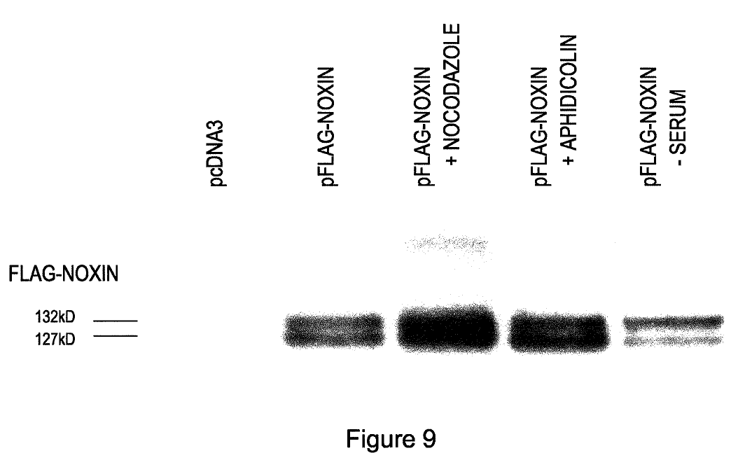

Upon transfection into cultured cells, the cloned cDNA generates a protein with electrophoretic mobility corresponding to that of the endogenous protein (FIGS. 4F, 6C, 8, and 9), as determined by western blot analysis. In FIG. 6C, the NIH3T3 cells treated with stress stimuli (SNAP, a cocktail of cytokines (TNF-α, IFN-γ, and IL-1β), γ-irradiation (γ-IR), UV-irradiation (UV-IR), adriamycin (ADR), or hydrogen peroxide (H₂O₂)) were analyzed for the Noxin protein by western blot using anti-Noxin antibody. Noxin appeared in three separate bands of 116, 125 and 129 kD. In FIG. 9, after transfection with pFLAG-noxin or pcDNA3 vector, cell division was arrested with three different treatments (nocodazole, aphidicolin, or low concentration of serum). Recombinant protein was detected on a Western blot using the anti-FLAG antibody. FLAG epitope increased the electrophoretic mobility of the recombinant protein.

### Example 5

### Plasmid Construction

The cDNA encoding the entire noxin polypeptide was excised from IMAGE clone A1157360 (Research Genetics) by XhoI and subcloned into pcDNA3 (Invitrogen). The 5′-end XhoI-HindIII noxin cDNA fragment was amplified by PCR using primers with a 2×FLAG tag sequence and was used to replace the original noxin sequences to generate the pFLAG-noxin expression plasmid. Plasmids expressing p53, p53dn (a dominant negative form of p53), and p21/WAF were gifts from Dr. S. Lowe (Cold Spring Harbor Laboratory). The pEGFP-N1, pCFP-nuc, and pYFP-actin were purchased from Clontech.

### Example 6

### Tissue Distribution of Noxin mRNA in Adult Mouse Tissue

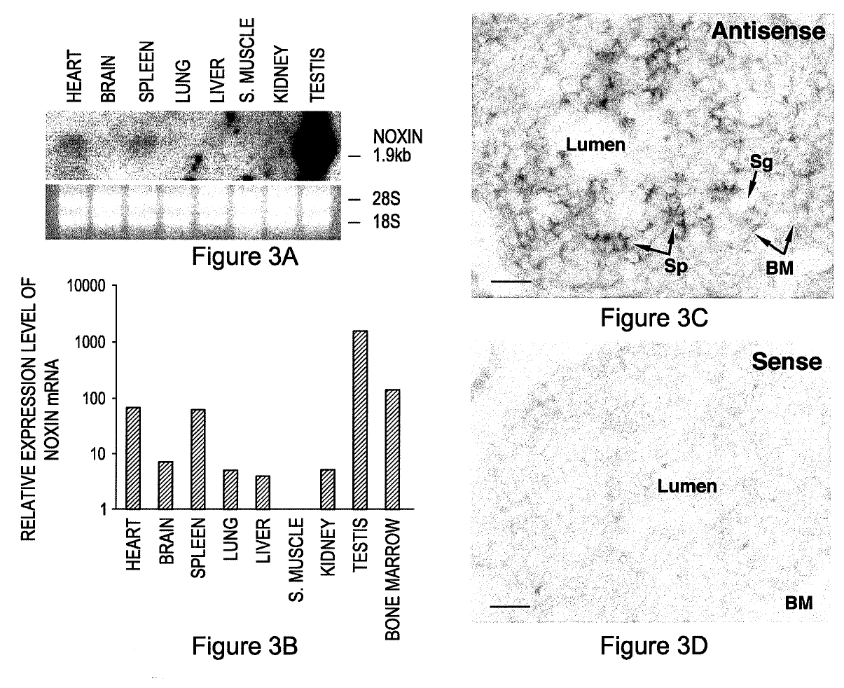

Total RNA was extracted from different adult mouse tissues and analyzed for noxin mRNA by Northern hybridization. Northern blot analysis was carried out as follows. Total RNA (10 μg) was separated by denaturing formaldehyde-agarose gel electrophoresis and transferred onto a nylon membrane (Hybond-N⁺, Amersham). The membrane was pre-hybridized with hybridization buffer (0.05 M sodium phosphate buffer, 5×SSC, 4×Denhardt's solution, 1% SDS, 50% formamide, 0.1 mg/ml salmon sperm DNA, and 0.2 mg/ml yeast tRNA) for 6 h at 42° C. and hybridized with the same buffer containing 100,000 cpm/ml [α-³²P] dCTP-labeled noxin cDNA probe amplified by PCR from pFLAG-noxin. Following hybridization for 20 h at 42° C., the membrane was washed twice with 2×SSC for 5 min at room temperature, twice with 0.2×SSC for 5 min at 65° C. and twice with 1×SSC with 3% glycerol for 5 min at room temperature. The membrane was exposed for 3 days at −70° C. using X-OMAT X-ray film (Kodak). Northern blot analysis of RNA from various mouse tissues showed that noxin is strongly expressed in the testis as well as in the spleen and heart (FIG. 3A).

These results were further confirmed by Real-time quantitative RT-PCR (Q-PCR), which was carried out as follows: total RNA (2 μg) was converted into cDNA by Taqman Multiscribe reverse transcriptase (125 U) (Applied Biosystems) using random hexamer primers. The cDNA was diluted, mixed with 2×SYBR Green PCR Master Mix (Applied Biosystems) and 0.3 μM forward and reverse primers, and amplified using the ABI PRISM 7700 Sequence Detection System (Applied Biosystems). Primers (Sigma Genosys) for noxin and β-actin were designed using Primer Express software (Applied Biosystems) assuming the Tm value of 58-60° C. and the amplicon size of 70-150 bp:

| | |
|---|---|
| 5′-TTTCTACCGGATACTTGCTCATCA-3′, | (noxin-FW; SEQ ID NO:7) |
| 5′-GTTGCAAGACCCCACTAGTCCT-3′, | (noxin-RV; SEQ ID NO:8) |
| 5′-CGTGAAAAGATGACCCAGATCA-3′ and | (β-actin-FW; SEQ ID NO:9) |
| 5′-CACAGCCTGGATGGCTACGTA-3′. | (β-actin-RV; SEQ ID NO:10) |

The PCR conditions for the thermal cycler were as follows: denaturation (1 cycle at 95° C. for 10 min), amplification (40 cycles at 95° C. for 15 sec and 60° C. for 1 min). During the amplification, the fluorescence of each sample in the plate was detected in real time and an amplification curve was drawn by Sequence Detection System Software (Applied Biosystems) to get the threshold cycle Cτ, the cycle number at which the curve starts to rise over the background noise. The data from noxin amplifications were normalized using the Cτ value from the β-actin set for each sample and the relative difference in the amount of noxin RNA in untreated and in stress-exposed cells was calculated.

Data of the Q-PCR analysis of noxin mRNA, where the data represent relative amounts of noxin mRNA compared to its levels in the skeletal muscle, showed the relative enrichment of the noxin mRNA in testis, bone marrow, spleen, heart, kidney, brain, lung and liver (FIG. 3B).

### Example 7

### Noxin Expression in the Testis

Strong expression of noxin in the testis was confirmed by in situ hybridization, immunocytochemistry, and Western blot analysis (FIGS. 3A-D and 4A-F). In FIG. 4F, a Western blot analysis, Noxin protein is expressed in the testis of the noxin^{+/+} but not of noxin^{-/-} animals; its expression is reduced in the noxin^{+/-} heterozygotes.

In situ hybridization. Adult mouse (C57BL/6) tissues were directly frozen in OCT compound (Tissue-Tek), sectioned by cryostat (10 μm thickness), and mounted on glass slides (ProbeOn Plus, Fisher). The noxin cDNA PCR fragment was subcloned into pCRII-TOPO. Digoxigenin (DIG)-labeled noxin RNA probes were transcribed from SacI-linearized constructs in vitro using T7 RNA polymerase (DIG RNA Labeling kit; Boehringer Mannheim) according to the manufacturer's instructions. Hybridized RNA was detected using alkaline phosphatase-conjugated anti-DIG according to the procedure described by (Decimo et al., In B. D. Hames, and S. J. Higgins (ed.), Gene Probes 2: A Practical Approach, vol. 2. IRL Press at Oxford University Press, New York 183-210 (1995)).

Male germ cells in the seminiferous epithelium of the testis are spatially and temporally arranged in a distinct pattern that enables their identification: the spermatogonia are localized along the basement membrane at the periphery of the tubules, whereas the primary spermatocytes are localized in the middle layers of the tubules, and the spermatids are found close to the lumen (FIGS. 3C, D). Furthermore, the distinct morphology of spermatocytes allows their discrimination among the other cell types in the tubules. Two images (FIGS. 3C, D) showing the noxin mRNA expression pattern in adult mouse testis visualized by in situ hybridization using DIG-labeled antisense noxin RNA probe (upper panel) and sense RNA probe (lower panel). The sense probe produced no signal. The hybridization images show that noxin mRNA was mainly expressed in primary spermatocytes. Scale bar: 25 μm.

Detection of noxin protein using anti-noxin antibodies. Anti-noxin antiserum was raised by immunizing a rabbit against a synthetic peptide having an amino acid sequence of the C-terminal region (870-886) of noxin (RKCLDLHYSPDPKELPR (SEQ ID NO:13)) using standard laboratory techniques. For immunohistochemistry, mice were perfused transcardially with PBS and then 4% paraformaldehyde in PBS at 4° C. Dissected testes were postfixed for 4 h and cryoprotected in 30% sucrose overnight at 4° C. Sections were cut on a cryostat (10 μm thickness) and collected on coated slides (ProbeOn Plus, Fisher). The slides were gradually dehydrated, air-dried, and stored at −20° C. Following rehydration, the slides underwent an antigen retrieval step: 10 min incubation at 95-100° C. in 0.01 M sodium citrate (pH 6.0), cooled to room temperature, and rinsed with distilled water. Slides were then incubated in 0.3% hydrogen peroxide to inactivate endogenous peroxidases, washed in PBS containing 0.1% Triton X-100 (PB S-T), and blocked at room temperature for 1 h in 3% normal goat serum in PBS-T. The slides were incubated overnight at 4° C. with primary antibody (anti-noxin antibody, 1:500) diluted in PBS-T with 1% goat serum. After washing with PBS-T, the slides were incubated with a biotinylated goat anti-rabbit secondary antibody (1:200; Vector Laboratories) for 2 h, followed by further washing and incubation in HRP-conjugated avidin-biotin complex (Vector ABC Elite) for 1.5 h before staining with 3,3′-diaminobenzidine and hydrogen peroxide. All slides were counterstained with hematoxylin and coverslips mounted with Permount (Fisher).

The immunocytochemistry using a polyclonal antibody to noxin further confirmed the noxin production in the testis (FIG. 4A-F). Noxin protein is highly expressed in the primary spermatocytes and, to a lesser extent, in the round spermatids (FIG. 4A). This staining is highly specific as the signal is absent in each of the following: in testis from noxin knockout animals (FIG. 4C), in the presence of the peptide used to raise the antibody, added as a competitor (FIG. 4D), or in the absence of the primary antibody (FIG. 4E). Antibody staining, particularly when visualized using Nomarski imaging (FIG. 4D), demonstrates that in primary spermatocytes noxin is localized to the periphery of the nucleus, in a structure which may correspond to the centrosome, the Golgi body, or the XY body of these cells.

Western blot analysis. Cells or tissues were homogenized in the lysis buffer (10 mM Tris-HCl pH 7.5, 1 mM EDTA, 400 mM NaCl, 0.5% NP40, 5 mM NaF, 0.5 mM sodium orthovanadate, 10% glycerol, 1 mM dithiothreitol, 1 mM PMSF, 1 μg/ml aprotinin, 1 μg/ml leupeptin and 1 μg/ml pepstatin) for 20 min on ice. Following centrifugation, the soluble fraction was collected and protein concentration was determined using the BCA assay kit (Pierce). 50 μg of total protein was separated on an 8% SDS-PAGE gel and transferred to Immobilon-P membrane (Millipore Corporation) by semi-dry blotting. The membranes were incubated with anti-noxin antibody (see above) (1:1,000) followed by anti-mouse IgG or anti-rabbit IgG antibody conjugated to horseradish peroxidase (HRP) (1:10,000, Amersham). The HRP signal was detected using a chemi-luminescence detection kit (SuperSignal Femto Dura Extended Duration Substrate, Pierce) and Hyperfilm X-ray film (Amersham Biosciences).

The expression of noxin in the testes was confirmed by Western blots, which showed noxin expression in the testis from wild type mice, absence of the protein in the knockouts, and an intermediate level of expression in heterozygous animals (FIG. 4F). In a and b, arrows and labels identify specific tissues and germ cell types: BM, basement membrane; Sg, spermatogonia; Sp, primary spermatocyte; Sd, spermatid. Scale bar: 25 μm.

### Example 8

### Subcellular Location of Noxin Protein in the Cell

To determine the subcellular localization of noxin protein, NIH3T3 cells were transfected with a plasmid encoding noxin tagged with the FLAG epitope at its N-terminus (pFLAG-noxin). The plasmid was prepared as described in Example 5. NIH3T3 cells grown on cover slips coated with collagen and poly-L-lysine were transfected with pFLAG-noxin. Cells were fixed with 4% methanol-free formaldehyde in PBS for 20 min on ice. After permeabilizing with 0.1% Triton X-100 in PBS for 10 min on ice, cells were incubated with primary and secondary antibodies in PBS (containing 0.05% Tween 20 and 1% BSA) each for 1 h at room temperature. The primary antibodies used in these experiments were anti-FLAG (1:500, Sigma), anti-p53 (1:1,000, Novocastra), and FITC-conjugated anti-BrdU antibody (1:500, Pharmingen). The secondary antibodies were ALEXA Fluor 488-conjugated goat anti-mouse IgG (1:500, Molecular Probes) and ALEXA Fluor 546-conjugated goat anti-rabbit IgG (1:500, Molecular Probes). Cell nuclei were counterstained with Hoechst 33342 (Molecular Probes) and the cover slips were mounted with Gel/Mount medium (Biomega). Fluorescence signals were analyzed under a fluorescence microscope (Zeiss Axiophot) equipped with Plan-Neofluar objectives (×40-100) and a CCD camera. Data were analyzed by Photoshop software (Ver. 6, Adobe).

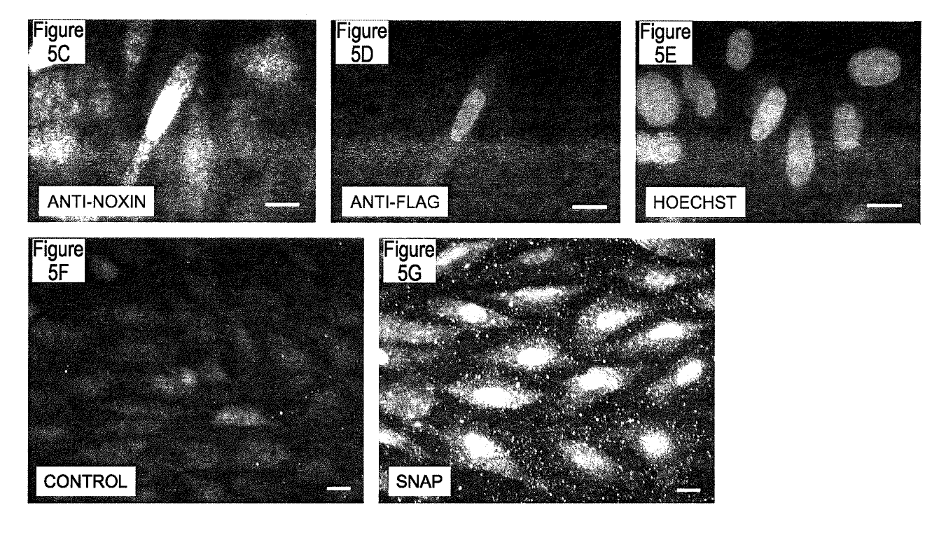

In FIG. 5, anti-FLAG antibody signal was seen as red fluorescence and Hoechst 33342 was seen in blue. Scale bar: 5 μm. A majority of the transfected cells showed nuclear localization (arrowhead) of noxin, compatible with the presence of two predicted nuclear localization signals in the noxin protein (FIGS. 5A,B); at the same time, in a number of cells, noxin was present in the cytoplasm (arrow) (FIG. 5A). FIG. 5C-E is a set of panels of fluorescent images showing the subcellular distribution of the transfected noxin protein. Staining (green) of pFLAG-noxin-transfected cells with anti-Noxin antibody (Panel 5C), anti-FLAG antibody (Panel 5D), or Hoechst33342 (Panel 5E). Scale bar: 5 μm. The distribution of the protein was identical, whether revealed by the polyclonal antibody to noxin or by the antibody to the FLAG epitope. FIG. 5F-G is a set of panels of fluorescent images showing the subcellular distribution of the transfected noxin protein. Panels 5F and 5G show that the expression of endogenous noxin, visualized with an antibody raised against a C-terminal region peptide, was mainly localized in the cytoplasm. Exposure to SNAP resulted in a predominantly nuclear localization of the protein. (Note that the quality of the endogenous noxin signal does not allow us to visualize its precise subnuclear distribution). When in the nucleus (FIG. 5G), noxin can be seen in subnuclear particles in the euchromatic regions and the protein accumulated in the nucleus when cells were exposed to the NO donor SNAP. These noxin-containing particles did not colocalize with several subnuclear structures revealed with antibodies to PML (a component of PML bodies), SF2 (a component of spliceosomes), or phospho-H2AX (a DNA repair-related protein) (data not shown).

### Example 9

### Noxin Expression is Induced in Response to Various Stress Stimuli

Noxin was identified as an NO-inducible gene and the time course of its induction by the NO donor SNAP was determined. Noxin mRNA levels as determined by Northern blot started to increase 2 h after the addition of SNAP and reached maximal levels 8 h after the addition (FIG. 6A). The pattern of noxin induction by SNAP was similar to that of a p53-dependent gene mdm2. Results for β-actin are shown as a control.

Noxin is induced not only by NO but also by other known stress stimuli. NIH3T3 cells were exposed for 16 hours to γ-irradiation (γ-IR), UV-irradiation (UV-IR), the chemotherapeutic agent adriamycin (ADR), hydrogen peroxide (H₂O₂), and a mixture of cytokines (TNF-α, IFN-γ, and IL-1β) which is known to be a potent inducer of reactive oxygen and nitrogen species. Each of these treatments resulted in a strong induction of noxin mRNA (e.g., 21-fold for the UV-irradiation) as determined by Q-PCR (FIG. 6B), demonstrating that the noxin gene can respond to a wide range of dissimilar stimuli by transcriptional activation, thus characterizing noxin as a general, stress-responsive gene.

In parallel to the noxin mRNA accumulation in response to the stress stimuli, noxin protein levels were also increased by each of the tested stimuli (FIG. 6C). In unstimulated cells, endogenous noxin protein is revealed on the Western blot using anti-noxin antibody, as a major band migrating at 116 kD and a minor band at 125 kD. All of the examined stimuli increased the noxin protein levels and some (e.g., SNAP, UV-irradiation, adriamycin, and H₂O₂) specifically increased the levels of the 125 kD polypeptide. Furthermore, in the SNAP-, cytokines-, γ-irradiation, and adriamycin-treated cells an additional band of 129 kD was evident. Together, these results demonstrate that noxin mRNA and protein expression is strongly induced by each of the tested stress stimuli and that a specific set of polypeptides (presumably, reflecting posttranslational modifications of the serine-rich noxin protein) is induced in response to each stimulus.

Noxin expression is induced both by chemical NO donors and by in vivo stimuli that induce NO production, e.g. cytokines. Noxin is also induced by a wide range of stimuli: γ-irradiation, UV-irradiation, adriamycin, and hydrogen peroxide. These agents activate different immediate targets, but converge on a small number of effectors which can slow down or halt the cell cycle, suppress cell death pathways, and permit the repair process to proceed. Alternatively, these effectors may activate the cell death program to eliminate defective cells.

### Example 10

### p53 is Necessary for Induction of Noxin by Stress Stimuli

Noxin induction by SNAP is prevented by the deletion of the p53 gene. The contribution of p53 to noxin induction was examined by comparing the effect of the stress treatments applied to the NIH3T3 cells, on noxin mRNA expression in mouse embryonic fibroblasts (MEFs) from wild type and transgenic animals lacking p53 (p53^{-/-} animals). MEFs isolated from wild type or noxin^{-/-} mice were incubated in low-serum medium (0.5% FBS) for 24 h to stop their cell cycle. The medium was then replaced with DMEM with 10% FBS to restart the cell cycle, and SNAP and BrdU were added to the medium. At the end of the incubation, BrdU-labeled cells were stained with FITC-conjugated anti-BrdU antibody and analyzed by flow cytometry. We have previously shown that in the absence of the p53 gene the induction of noxin by SNAP is reduced in NIH3T3 cells. (Hemish et al., J Biol Chem 278:42321-9 (2003))

MEFs expressing p53 (p53^{+/+}) and MEFs lacking p53 (p53^{-/-}) were exposed to stress stimuli, and analyzed for noxin mRNA expression (FIG. 6D, lanes arranged in the same order as in 6B). Similar to the NIH3T3 cells, each of these treatments increased the levels of noxin expression and, in each case, this induction was abrogated by the lack of p53 (FIG. 6D). This confirms the stress responsive nature of the noxin gene and demonstrates that induction of noxin in response to stress is dependent on p53.

### Example 11

### Noxin Expression is Regulated by the Cell Cycle

The relationship between cell cycle and noxin expression was examined by determining the noxin mRNA levels in cells in various phases of the cell cycle using the methodology as follows: NIH3T3 cells, synchronized by serum starvation (DMEM with 0.5% serum for 24 h) were released from arrest by the addition of 10% serum (corresponds to the 0 h time point). For comparison, asynchronized cell culture was used. Noxin mRNA expression was analyzed using Q-PCR 0, 4, 14, or 20 h after the addition of serum. The cell cycle phase of the cells was determined by flow cytometry in the following manner:

### Flow Cytometry.

The cells were labeled with 8-Bromo-deoxyuridine (BrdU) (10 μM) and incubating for the time periods indicated. The cells were collected from plates by trypsin digestion were suspended in saline and fixed with 70% ethanol. After washing with PBS containing 0.05% BSA, 1×10⁶ cells were incubated with FITC-conjugated anti-BrdU antibody (Pharmingen, 1:500) for 1 hr, suspended in PBS-BSA with 5 μg/ml propidium iodide (PI) and 20 μg/ml RNase A, and incubated further for 30 min at room temperature. For the bivariate cell cycle analysis, cells were analyzed using LSRII flow cytometer (PerkinElmer). For the detection of p53 by cytometric analysis, pFLAG-noxin- or pEGFP-transfected cells were fixed with 4% paraformaldehyde, permeabilized with saponin, and stained with primary antibodies (CM5 anti-p53 polyclonal antibody and Ml anti-FLAG monoclonal antibody, both from Sigma) and then with secondary antibodies (PE-conjugated goat anti-rabbit IgG and FITC-labeled goat anti-mouse IgG). Cells were analyzed for PE, FITC, and GFP fluorescence by flow cytometry.

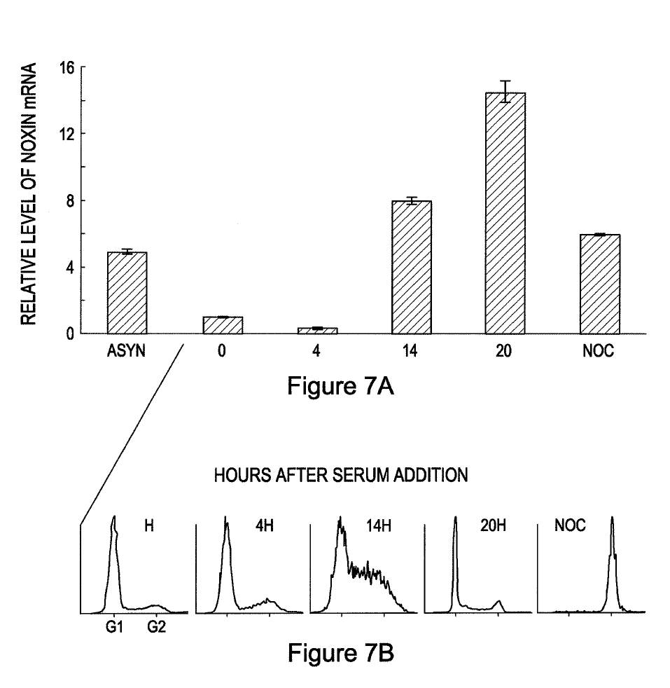

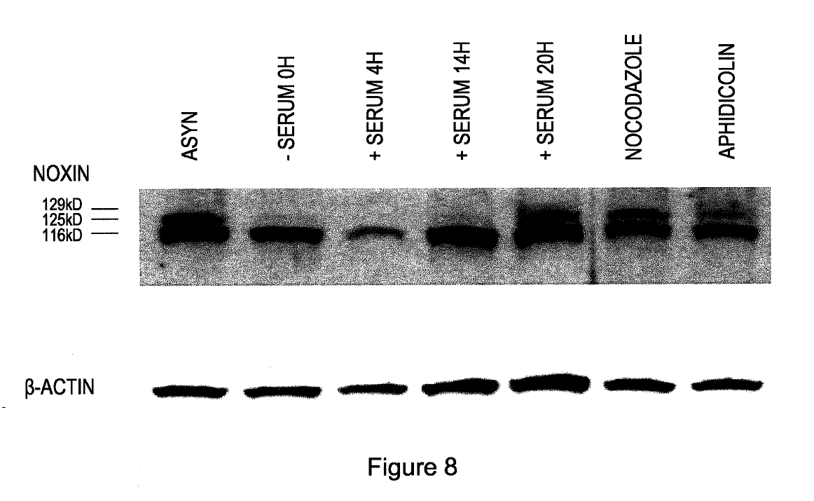

When NIH3T3 cells were arrested in G₀ phase by serum withdrawal, noxin mRNA expression was strongly reduced as compared to asynchronously dividing cells (FIG. 7A). Asyn: cells growing asynchronously. When cells were released from cell cycle arrest by the addition of serum, noxin mRNA was strongly induced (up to 15 fold) as the cells passed through S and G₂ phase. Noc: G2/M-arrested cells after treatment with nocodazole for 20 h. (the status of the cell cycle was determined by flow cytometry as seen in FIG. 7B. Flow cytometry confirmed that cells progress through the cell cycle phases (0-20 h time points) or accumulate in G2/M (nocodazole)). The cell cycle-dependent expression was also evident for the noxin protein; its levels were lower in the arrested cells than in the asynchronous culture, and strongly increased after the addition of serum and progression through the cell cycle (FIG. 8). The Noxin levels were highest in cells in G₂/M phase; they were also induced by cell cycle inhibitors nocodazole and aphidicolin (note that the levels of the minor 125 kD and 129 kD bands were selectively increased in response to serum or the inhibitors). Noxin expression was higher in cells treated with nocodazole (which arrests cells in G₂/M phase) than in cells exposed to aphidicolin or serum withdrawal (which induce S- and G₀-phase arrest, respectively).

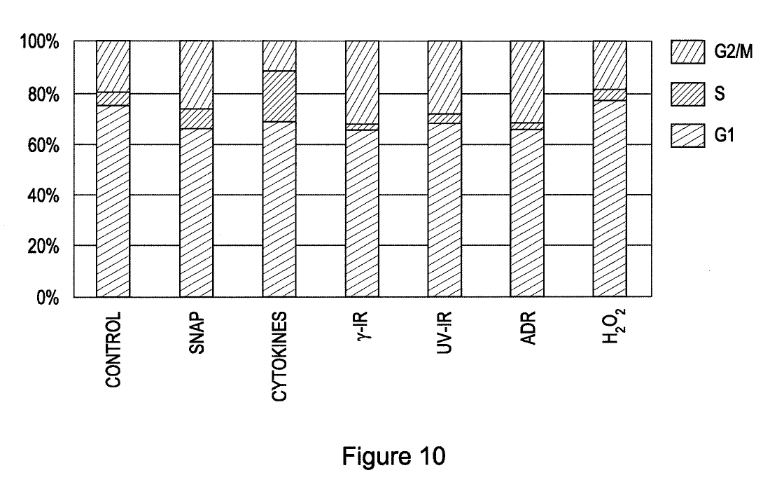

Although high levels of noxin mRNA and protein expression in serum- and nocodazole-treated cells in G₂/M phase raised a possibility that the stress-induced expression of noxin (FIG. 6A-D) merely reflected accumulation of cells in the G₂/M phase, this was not the case. Examination of the cell cycle phase-distribution of NIH3T3 cells exposed to each of the stress stimuli used for the experiments in FIG. 6 showed a) that each of these stimuli induced a specific pattern of cell cycle phase distribution and b) that there was no correlation between the level of noxin mRNA induction in response to a certain stimulus and the proportion of cells accumulating in G₂/M phase in response to this stimulus (for instance, the fraction of G₂/M cells after exposure to γ-irradiation is 2.75 and 1.75 fold higher than after exposure to cytokines and H₂O₂, respectively, whereas mRNA levels are 1.5 and 2.5 fold lower; furthermore, the proportion of G₂/M cells following H₂O₂ exposure is similar to that of unstimulated cells, yet the mRNA level is 13-fold higher) (FIG. 10). Together, these results demonstrate that noxin expression is controlled by the cell cycle and that noxin may interact with the cell cycle machinery. 10: Cells were fixed with 70% ethanol for 16 hrs after stress treatment and the cell cycle distribution was determined by PI staining and flow cytometry analysis. Segments of the bar depicted the proportion of cells in G1 (black), S (white), and G2/M (grey) phases of the cell cycle.

### Example 12

### Noxin can Induce Cell Cycle Arrest

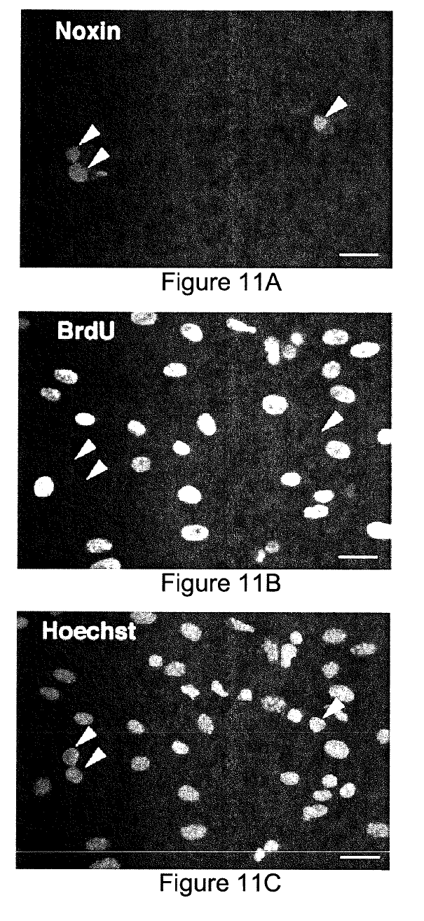

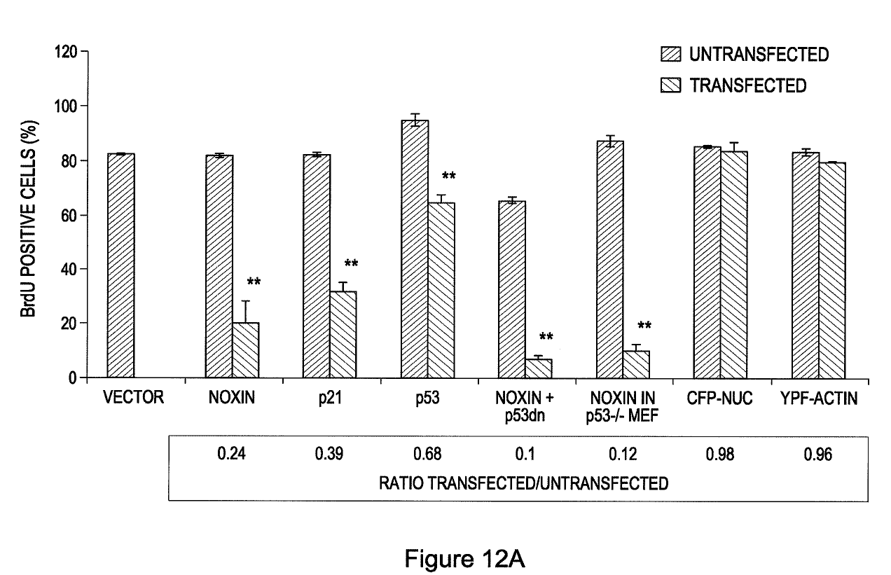

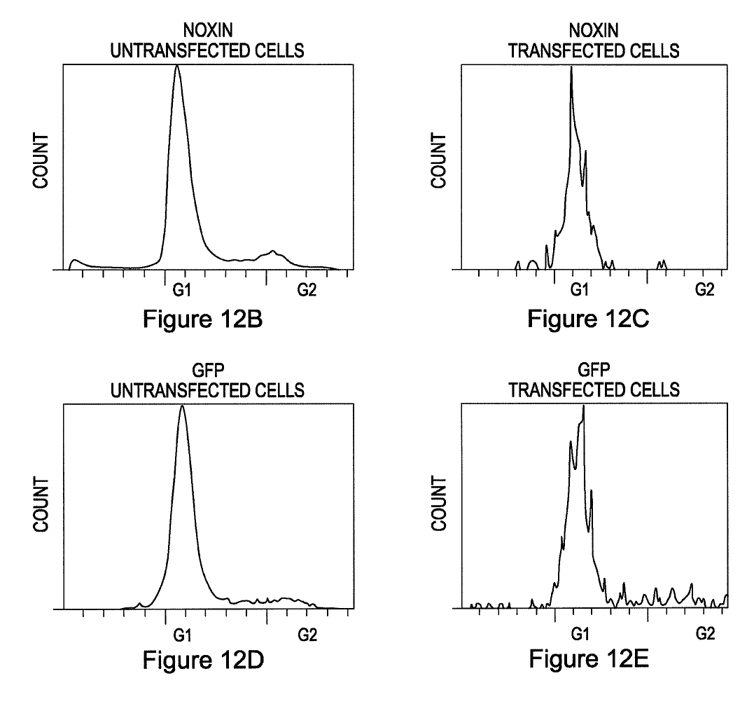

Conversely to the results presented in Example 11, noxin is not merely induced by cell cycle arrest but can induce cell cycle arrest, as determined in the following manner. NIH3T3 cells were transfected with plasmids encoding FLAG-noxin, p21/WAF, p53, or a dominant negative form of p53 (p53dn). In addition, constructs coding for cyan fluorescent protein with a nuclear localization signal (CFP-nuc), yellow fluorescent protein fused to actin (YFP-actin), or vector construct alone were used as controls. Cells were then labeled with BrdU for 24 h and analyzed cells in situ by immunocytochemistry for BrdU and noxin (or other listed proteins) (FIG. 11A-C). In the figures, signals from anti-FLAG antibodies are seen in red; anti-BrdU antibodies are green; Hoechst 33342 (blue) was used for nuclear counter-staining. The fraction of BrdU-positive cells were determined from among the cells that expressed the recombinant protein (transfected cells) and from among the cells that did not receive the transgene and did not express the recombinant protein (untransfected cells). As shown in FIG. 12A, fractions of BrdU-positive cells among the cell populations that were determined for cells which expressed or did not express transfected gene. Similar experiments were performed using constructs for p21/WAF, p53, dominant negative version of p53 (p53dn), cyan fluorescent protein with nuclear localization signal (CFP-Nuc) and yellow fluorescent protein-actin fusion (YFP-actin). Ratios of these fractions for transfected and untransfected cells are shown below the graph. Recombinant protein was detected on a Western blot using the anti-FLAG antibody. FLAG epitope increased the electrophoretic mobility of the recombinant protein. Comparison of these fractions shows that noxin was highly effective in suppressing DNA synthesis in transfected cells (24% of the value for the untransfected cells) (FIG. 12A). Both p21/WAF and p53 were also effective in suppressing DNA synthesis in cells that received these genes (39% and 68% of the values for the untransfected cells, respectively), whereas CFP-nuc and YFP-actin (taken as controls) did not show any effect on DNA synthesis in transfected cells (98% and 96%, respectively).

To determine the phase of the cell cycle affected by noxin, the NIH3T3 cells were transfected with pFLAG-noxin- or GFP-expressing recombinant plasmids and used flow cytometry following anti-FLAG staining and GFP fluorescence to compare the DNA distribution in cells that expressed or did not express the noxin transgene (FIG. 12B-C). After transfection with pFLAG-noxin or pcDNA3 vector, cell division was arrested with three different treatments (nocodazole, aphidicolin, or low concentration of serum). Cells were stained with anti-FLAG antibody and FITC-conjugated anti-mouse IgG antibody after permeabilization. Cell cycle distributions of untransfected and transfected cells were analyzed by DAPI staining and flow cytometry.

Most of the pFLAG-noxin-transfected cells had a 2n DNA content, whereas untransfected cells showed the usual distribution, with a fraction of cells having a 4n DNA set. In control experiments, cells that received or did not receive the GFP construct upon transfection, did not show any changes in the cell cycle distribution pattern showing that cells which express the transfected noxin gene are arrested in G₁ or early S phase.

Thus, in addition to its ability to control cell cycle progression, noxin is itself controlled by the cell cycle: the amount of noxin mRNA is very low in resting cells, but increases almost 50 fold as cells progress through S phase. In addition, its levels are decreased when cells are arrested by serum deprivation. Furthermore, noxin mRNA is mainly expressed in tissues which contain actively proliferating cell populations, such as bone marrow, spleen, and testis; also, it is expressed at much higher levels in actively dividing NIH3T3 cells than in slowly dividing MEF cells. The presence of noxin may be required in dividing cells to help protect them from stress and damage.

### Example 13

### p53 is Not Necessary for Noxin's Cell Cycle Arrest Activity

Although p53 is necessary for the induction of noxin under stress condition, the activity of noxin to affect cell cycle is not dependent on p53, as determined in the following manner. When noxin was cotransfected with a dominant-inhibitory version of p53, noxin still induced cell cycle arrest. Noxin action was also analyzed in p53-deficient MEFs. In accordance with the results of the dominant negative p53 expression, noxin was effective in suppressing DNA synthesis in MEFs from p53-deficient mice. Together, this shows that noxin expression can induce cell cycle arrest and that this action of noxin does not require the activity of p53.

Thus, expression of noxin elevates the cellular levels of p53 and is itself induced in a p53-dependent manner; however, noxin does not require p53 to induce cell cycle arrest. The possible role of noxin as a component of the cellular stress response system is further supported by its ability to induce cell cycle arrest (FIG. 11-12). Ectopic expression of noxin is as, or more, effective than expression of cell cycle inhibitors p21/WAF and p53 in inducing cell cycle arrest. It forces the cells to stall in G₁/S, perhaps helping the cells to activate their repair systems. Noxin was effective in inducing cell cycle arrest even in the absence of p53 activity (in p53^{-/-} MEFs or when co-transfected with a dominant negative p53 construct), indicating that it acts downstream or independent of p53.

### Example 14

### Noxin Induces p53

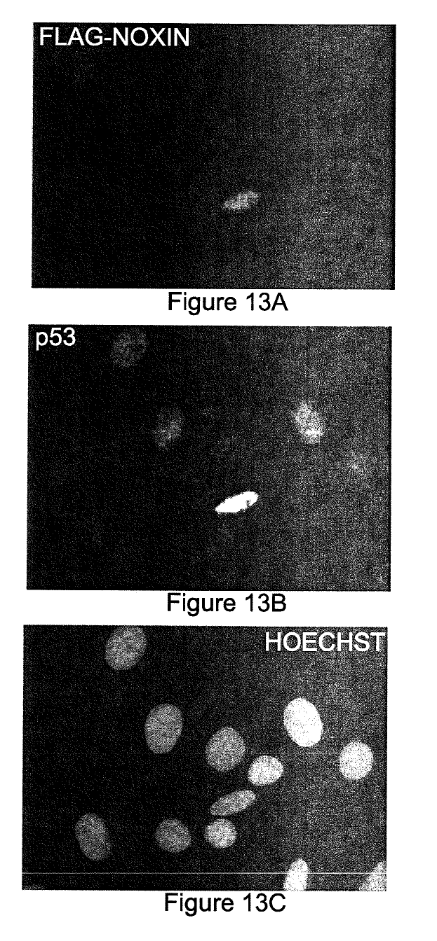

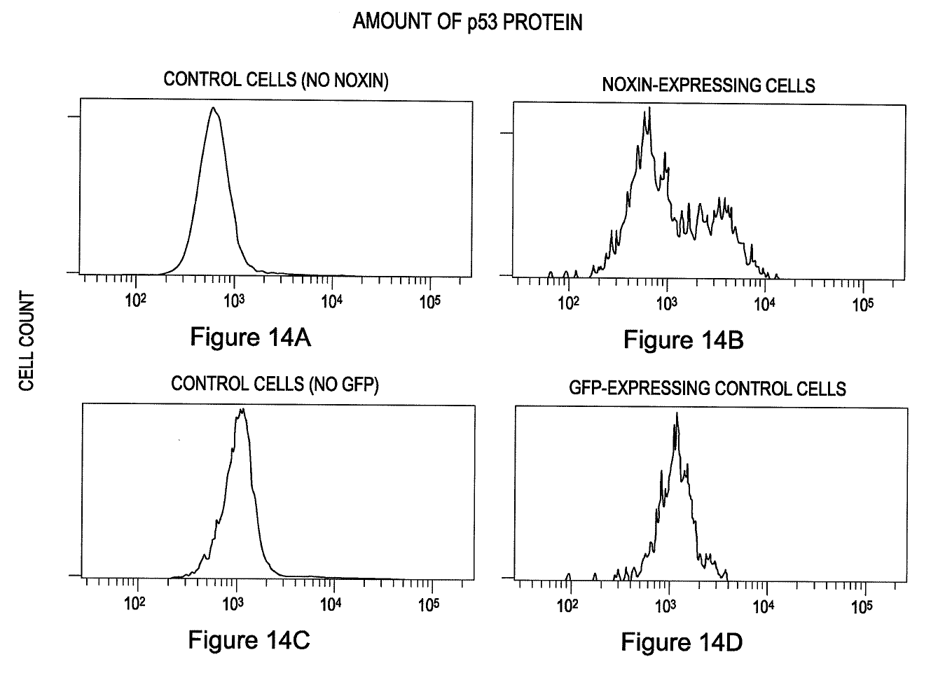

Cells were transfected with pFLAG-noxin- or GFP-expressing plasmids and the levels of p53 were determined using immunocytochemistry and flow cytometry. The p53 levels were increased only in the cells that received the noxin gene, but not in cells that did not show expression of noxin or in cells that received the GFP gene (FIGS. 13, 14). 13a-c: which were stained with anti-FLAG and anti-p53 primary antibodies and ALEXA Fluor 596- and 488-conjugated secondary antibodies, respectively. Cells expressing noxin (red) show higher levels of p53 (green) than cells that do not express noxin. 14a-d: Cells transfected with pFLAG-noxin (upper panels) and pEGFP (taken as control; lower panels) that were stained with anti-FLAG and anti-p53 primary antibodies, and PE- and FITC-conjugated secondary antibodies. Then Noxin-FLAG- or EGFP-negative (left panels) and positive (right panels) cells were analyzed for the amount of p53 protein.

Thus, noxin induction is dependent on p53 and that noxin can, in turn, induce p53 levels. This suggests a model where stress stimuli, in a p53-dependent manner, induce expression of noxin, which then induces cell cycle arrest and elevates the levels of p53; the latter, however, is not required for the antiproliferative action of noxin.

### Example 15

### Knockout of the Noxin Gene in Mice

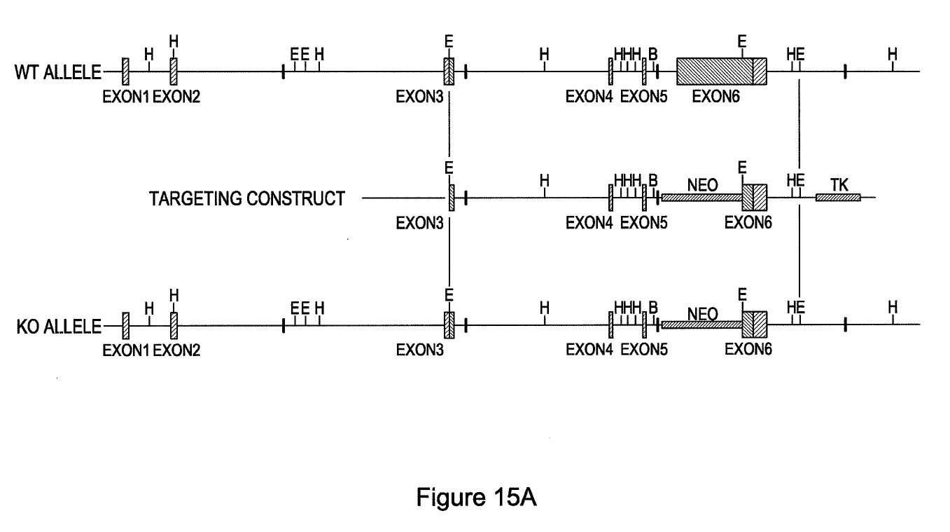

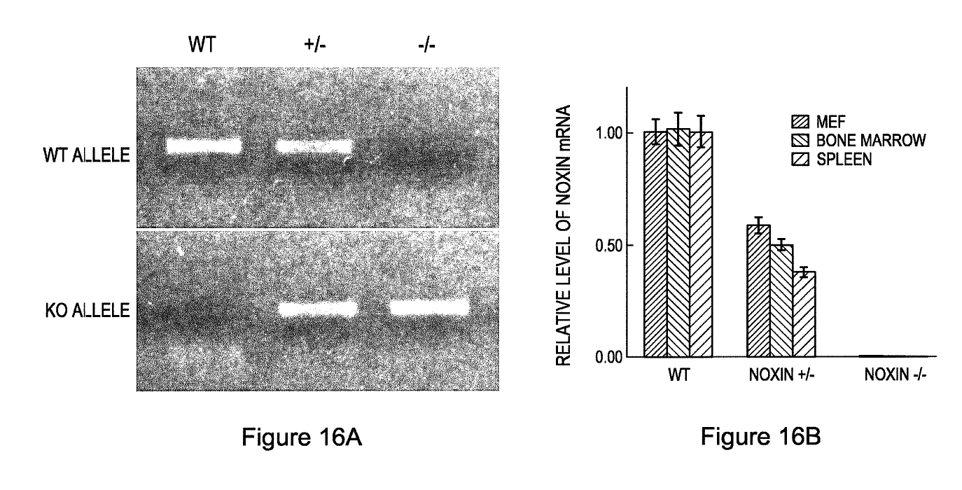

Noxin knock-out mice were created as follows: Exon 6 of the noxin gene was targeted since a large part of murine noxin is encoded by this exon (FIGS. 15, 16A). The bacterial artificial chromosome (BAC) containing the entire noxin gene was obtained by screening the BAC library with the noxin cDNA clone. A part of exon 6 (the longest exon) in noxin gene was replaced with a cassette containing a neomycin-resistance gene. (B: BamHI, E: EcoRI and H: HindIII). The resulting BAC clone was digested with EcoRI and HindIII to isolate the left and right arms of the targeting construct, respectively. After digestion and gel separation, 2 kb and 3 kb fragments were subcloned to flank both sides of the neomycin-resistance gene in the pPNT vector, generating the noxin targeting vector pPNT-noxin.

Cultured embryonic stem (ES) cells were transfected with pPNT-noxin and selected for neomycin-resistant clones by treatment with G418. Four hundred neomycin resistant clones were screened by nested PCR and further by genomic Southern blots. Two of the seven positive ES clones were injected into mouse blastocysts and mice were generated using standard techniques. Mice were genotyped by PCR using primer sets specific for the wild type and the knockout alleles. Replacement of these sequences with the neomycin phosphotransferase gene inactivated the noxin gene such that noxin mRNA could not be detected in the tissues of mice carrying a homozygous deletion of the gene or in the embryonic fibroblasts derived from these knockout mice. FIG. 16A is a Southern blot image showing the analysis of genomic DNA of mutant mice by PCR. using specific primer sets corresponding to the eliminated exon 6 (WT) and to the targeting cassette (KO). Wild type or knockout mice carry only one type of allele (WT or KO), while heterozygote mice have both alleles. Panel 16B shows the analysis of total RNA extracted from MEFs, adult bone marrow, and spleen for the expression of noxin mRNA by Q-PCR. Mice carrying one mutant noxin allele produced approximately half of the mRNA amount of the wild type animals. Tissues from noxin^{+/-} (heterozygous) mice expressed approximately half the amount of noxin mRNA of wild type mice. Noxin mRNA was not detected in tissues from noxin^{-/-} mice.

### Example 16

### The Phenotype Analysis of the Noxin Knockout Mice

Noxin heterozygotes produced homozygous mutant progeny at a normal Mendelian ratio, and the homozygous animals were born with a normal ratio of male to female mice. Homozygous noxin mutants were viable and fertile, developed without growth retardation, and displayed no detectable abnormalities on visual inspection and macroscopic analysis of the visceral organs. One notable exception was the heart which was larger in the knockout animals. See Table 1.

TABLE 1
Tissue weights and parameters in testis from wild type 1 and noxin⁻/⁻
mice.

| | | | | Parameters in testis | | |
|---|---|---|---|---|---|---|
| | Tissue Weight^{a} | | | Seminiferous Tubule | | |
| Genotype | Heart | Spleen | Testis | Diameter (μm) | Spermatocytes^{b} | Spermatids^{b} |
| Wild | 4.32 + 0.10 | 2.70 + 0.17 | 2.83 + 0.20 | 235.30 + 2.66 | 54.51 + 1.00 | 115.60 + 1.94 |
| type | | | | | | |
| Noxin−/− | 4.87 + 0.18* | 2.88 + 0.28 | 2.48 + 0.11 | 230.49 + 2.69 | 52.18 + 1.06 | 107.65 + 1.98** |

^{a}Milligram weight of organ per gram mouse body weight.
^{b}Number of cells in a cross section of tubule.
Shown are values ± standard errors.
*p < 0.05,
**p < 0.01.

### Testis

Because noxin was strongly expressed in testes (see Example 6), the structure of testes were examined in noxin deficient animals in the following manner: Immediately after euthanizing the adult mice (n=6 for wild type, n=7 for noxin^{-/-} mice), their testes were removed, fixed by 2% glutaraldehyde and subsequently embedded in Epon. Next, semi-thin sections (1 μm) were cut on a microtome (Reichert Ultracut E, Leica, Nussloch, Germany) and stained by toluidine blue-pyronine. To analyze potential differences in spermatogenesis between wild type and knockout mice, cross-sections of seminiferous tubules displaying the same stages of spermatogenesis were analyzed. Intact tubular cross sections of stage VI of spermatogenesis (n=93 for wild type, n=77 for noxin^{-/-} mice) were selected and the tubular diameter measured, which correlated with strong disturbances of spermatogenesis. The number of spermatocytes I and round spermatids visible in these tubules were counted. Data are presented as means±SEM of all seminiferous tubules analyzed. Statistical analysis was performed using SPSS Base 12.0 (SPSS software, Munich, Germany). Differences among experimental groups were analyzed with Mann-Whitney test as installed in SPSS Base, with p<0.05 being considered significant and p<0.01 highly significant.

Testis of noxin-deficient animals did not show detectable differences in weight, diameter of seminiferous tubules, or number of primary spermatocytes. However, there was a significant decrease in the number of round spermatids in noxin-deficient animals (Table 1).

### Blood

Noxin was also clearly detected in bone marrow and spleen (see Example 6). Therefore, these tissues were examined in noxin-deficient mice for abnormalities. The composition of blood and the content of hematopoietic progenitors in the bone marrow and spleen of homozygous knockout animals and their heterozygous and wild type littermates showed no significant differences in the blood cell profile in peripheral blood, spleen, or bone marrow, in the cellularity of spleen or bone marrow, in the number of CFC-8d and CFC-12d cells in the bone marrow, or in the number of CFC-12d colonies in the spleen. There were detectable differences in the number of CFC-8d cells in the spleen of homozygous knockouts (12% increase, p<0.05) and Sca1-positive cells in the bone marrow (a 45.5% decrease, p<0.001). Thus, there were only a few significant differences that were detectable between the wild type and knockout mice in a wide range of hematopoiesis-related parameters.

### Toxic Shock

The response of noxin mutant mice to lipopolysaccharide (LPS)-induced endotoxic shock was tested using a standard method in the art. LPS challenge induces oxidative and nitrosative stress and, at high doses, results in mortality. The response of the wild-type noxin^{+/+} mice was compared with that of their littermates carrying a homozygous or a heterozygous deletion of the noxin gene, and no differences in mortality between the three genotypes were found. It is possible that the threshold for detecting the differences between the wild type and noxin knockout mice has not been achieved.

Contrary to the clear consequences of the loss of the noxin gene in cultured cells, in knockout animals only small changes (e.g., heart size, the level of Sca1⁺ cells, and the number of round spermatids) are seen, and deleting the gene on how the animals respond to stress, e.g., to endotoxic shock or for the maintenance of the tissues and organs where it is expressed at high levels (testis, spleen, bone marrow) is unknown. Consistent with the observation that noxin is selectively expressed in primary spermatocytes, these cells are more resistant to NO-induced apoptosis than other types of cells involved in spermatogenesis, e.g., spermatids (Di Meglio et al., Biochim. Biophys. Acta 1692:35-44 (2004)). Added to the observation that in the knockout animals the number of TUNEL-positive cells in the seminiferous tubules is increased (data not shown) and is accompanied by a decrease in the number of spermatids, noxin may be considered to contribute to the protection of the germline cells from the nitrosative stress or other types of insults.

### Example 17

### Absence of Noxin does not Affect DNA Synthesis or NO Response

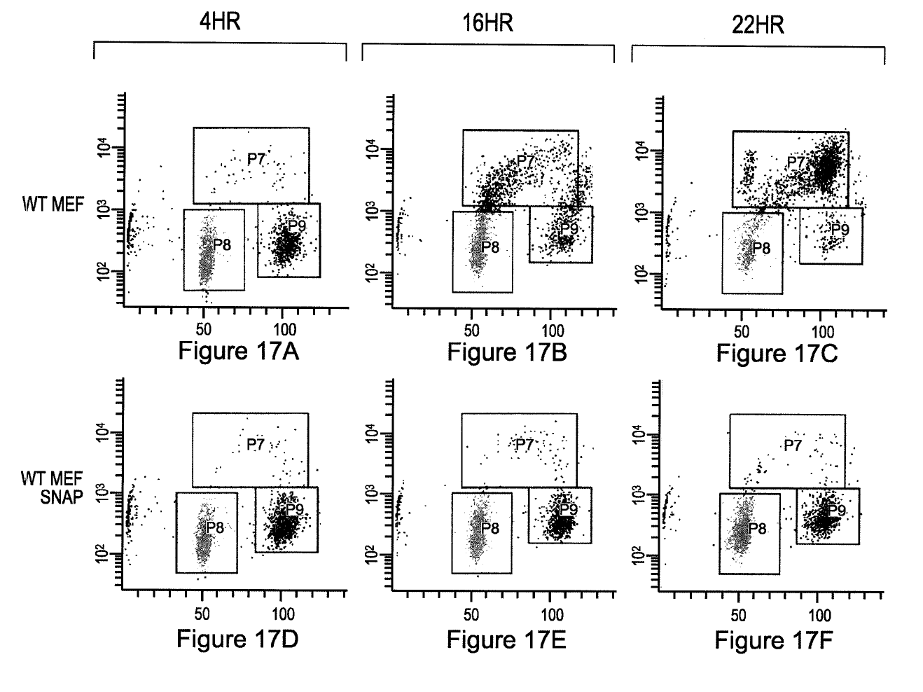

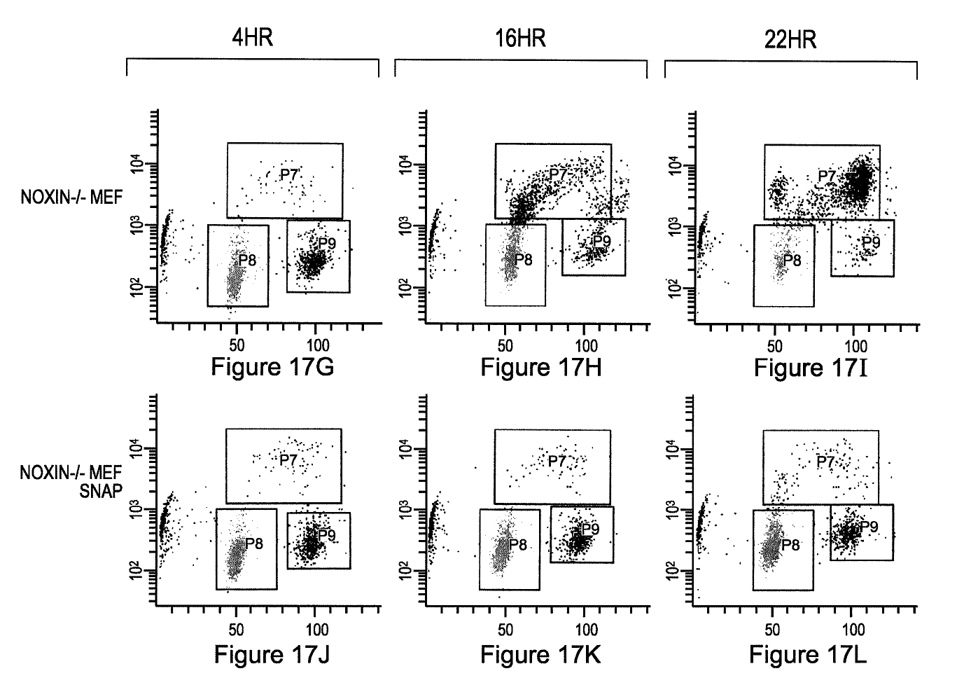

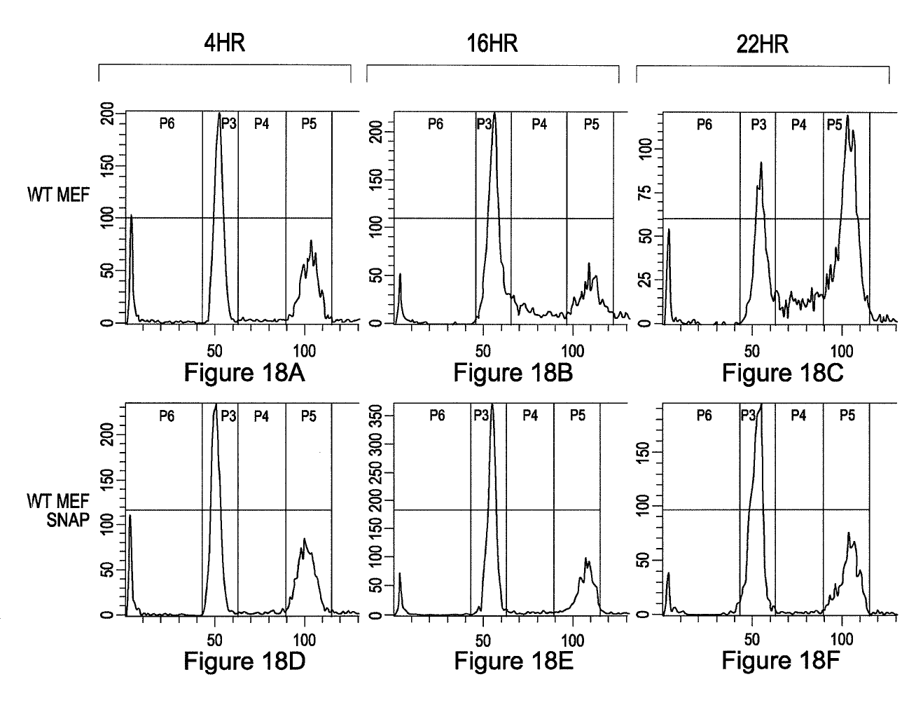

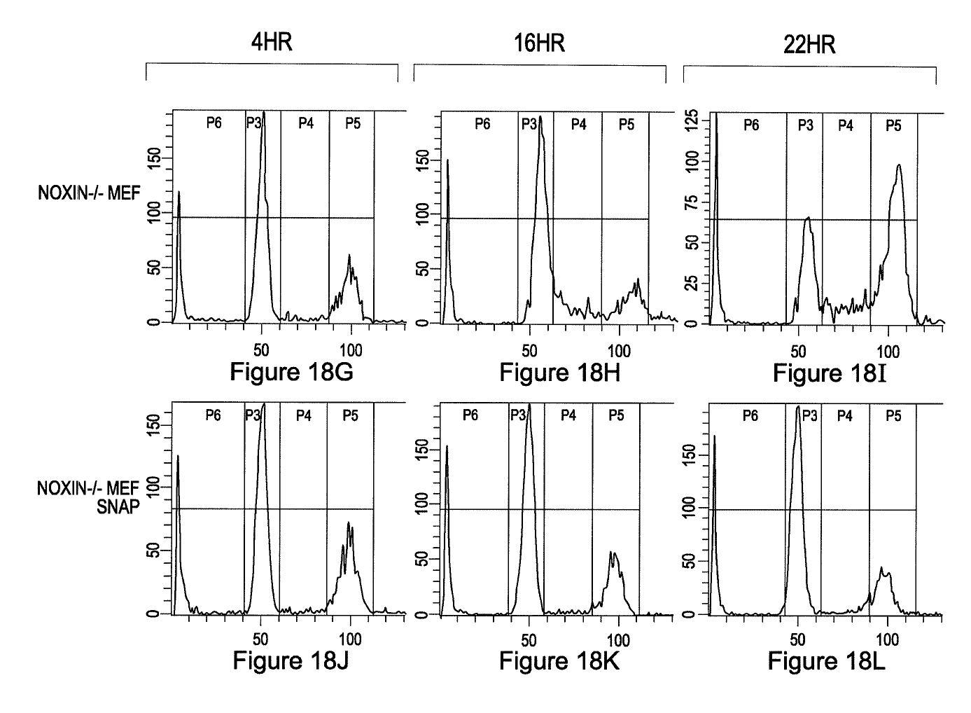

Since noxin can induce cell cycle arrest and, in addition, respond to various types of stress stimuli, including NO, the growth characteristics of MEFs isolated from noxin^{-/-} animals or their wild type (noxin^{+/+}) littermates were compared. MEFs from wild type and noxin^{-/-} mice were arrested with low serum (0.1%) culture medium for 24 h and released by adding 10% serum. Cells were either left untreated or exposed to SNAP. At the time of release, BrdU was added to the culture medium and cells were further incubated for 4, 16, or 22 h (FIG. 17, indicated). Cells were collected, stained with PI and analyzed by flow cytometry for BrdU-positive cells (FIG. 17, X-axis: PI, Y-axis: BrdU) and for cell cycle distributions (FIG. 18, X-axis: PI and Y-axis: number of cells) (Fraction P3=G₁, Fraction P4=S, Fraction P5=G₂/M and Fraction P6=Sub-G₁ populations). The analysis provided detailed information on how cells progress through the cell cycle, with a particular emphasis on the S phase. The effect of SNAP on cells was dramatic, decreasing the fraction of dividing cells by 95.5%; however, no differences were detected between the responses of the wild type vs. the mutant cells., when analyzing either the fraction of cells in the S phase or the kinetics of response to SNAP (compare panels b to e, c to f for wild type, and 17h to k and i to l for noxin^{-/-} of FIGS. 17 and 18). Thus, inactivation of the noxin gene does not noticeably affect the rate of DNA synthesis or the response of cells to NO.

### Example 18

### Cell Death is Increased by Noxin Gene Ablation or Down Regulation—Noxin^{-/-} MEFs Experiment

The growth of noxin^{-/-} MEFs under normal conditions (FIG. 17g-i) and after addition of SNAP (FIGS. 17j-l) were similar to that of wild type MEFs (FIG. 17a-c and FIG. 17d-f). However, the fraction of sub-G₁ cells was higher in noxin-deficient (noxin^{-/-}) cells (Fraction P6) at each time point analyzed than in wild type (noxin^{+/+}) MEFs (13.8, 16.3, and 11.5 vs. 9.7, 5.1, and 3.5%, respectively, FIG. 18 fraction P6). Incubation with SNAP did not noticeably affect this fraction in the noxin^{+/+} or noxin^{-/-} cells. Since the sub-G₁ fraction corresponds to dying cells which have lost part of their DNA, these data suggest that lack of noxin increases the fraction of cells which undergo apoptosis.

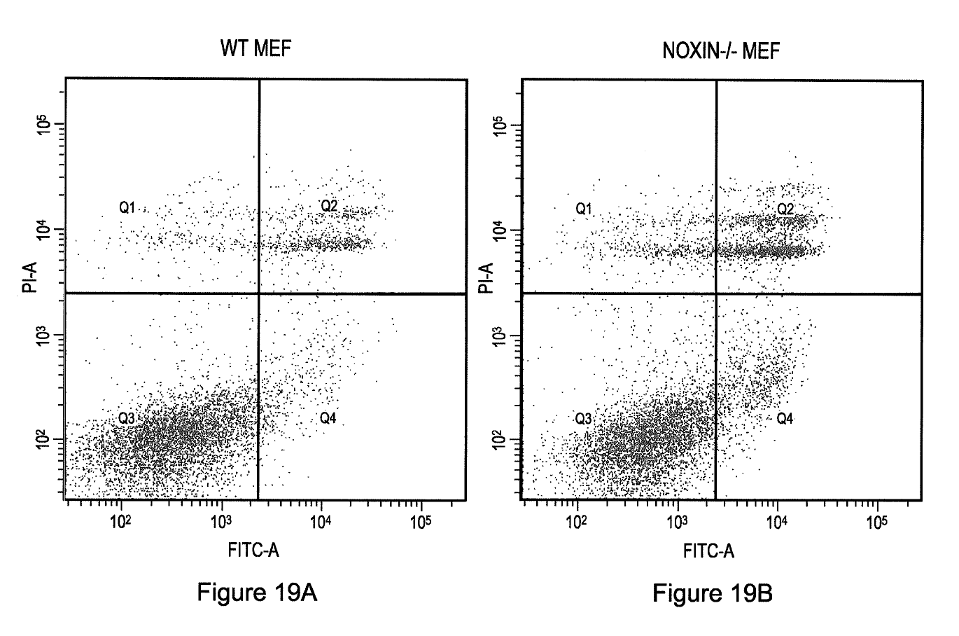

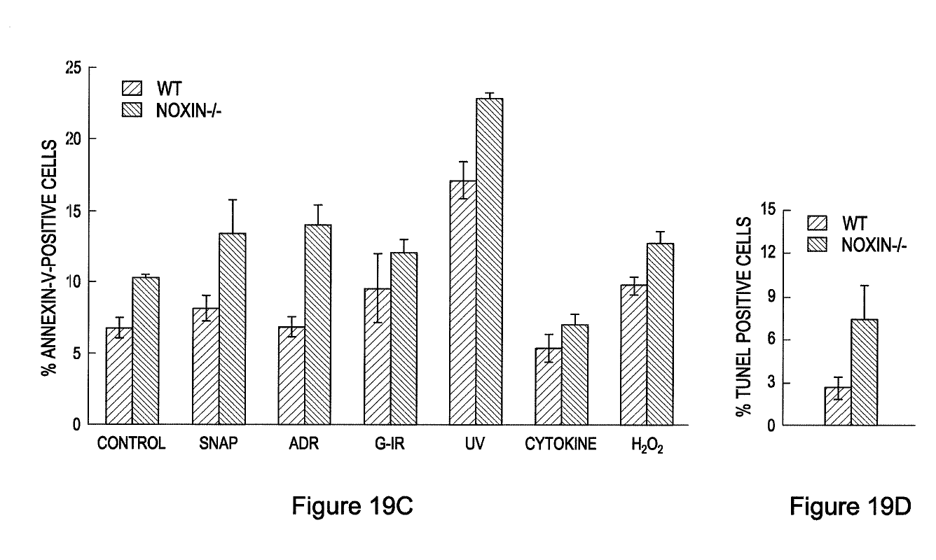

This was tested further by using alternative assays for apoptosis, measuring annexin V⁺ PI⁻ cells (FIGS. 19A-C) and TUNEL-positive cells (FIG. 19D) among noxin^{+/+} and noxin^{-/-} cells. To detect apoptosis, MEFs isolated from wild type or noxin^{-/-} mice were grown on cover slips coated with collagen and poly-L-lysine, in normal culture medium or treated with the previous panel of stress stimuli (SNAP, cytokines, UV-irradiation, γ-irradiation, adriamycin, or hydrogen peroxide) for 16 h. Cells were collected and assayed for annexin V positive cells (FIG. 19A-C). Wild type and noxin^{-/-} MEFs were also grown in culture, washed with PBS, and stained with a FITC-conjugated annexin V antibody (Molecular Probes) and PI according to manufacturer's instructions. Annexin V⁺ PI⁻ cells were quantified by flow cytometry (FIG. 19A-B, X-axis: annexin V, Y-axis: PI) (Q1: annexin V⁻ PI⁺, Q2: annexin V⁺ PI⁺, Q3: annexin V⁻ PI⁻, Q4: annexin V⁺ PI⁻). Examples are shown for untreated wild type MEFs (Panel 19A) and noxin deficient MEFs (Panel 19B). The graph (Panel 19C) shows the percentage of annexin V⁺ PI⁻ cells (Q4) for each of the stress stimuli tested.

TUNEL-positive cells were quantified by microscopy. The cells were washed with PBS, and processed for the terminal deoxynucleotidyltransferase-mediated dUTP-fluorescein nick end labeling (TUNEL) assay according to instructions in the ApoAlert DNA Fragmentation Assay Kit (BD Biosciences). The number of TUNEL positive cells is expressed as a percentage of the total cell population. Data shown in bar graphs represents the means±standard error (Panel 19D).

The results of these assays show that noxin^{-/-} MEFs had significantly larger numbers of apoptotic cells (140% by the annexin assay and 285% by the TUNEL assay) than their wild type counterparts (FIGS. 19C, D).

Furthermore, the annexin assay for apoptosis were applied to noxin^{+/+} MEFs subjected to the panel of treatments described above: SNAP, γ-irradiation, UV-irradiation, adriamycin, hydrogen peroxide, and cytokines. For each type of stress, the fraction of apoptotic cells was higher in noxin-deficient MEFs than in their wild type counterparts (FIG. 19C). Together, these three assays (DNA content measurement, TUNEL, and annexin V/PI flow cytometry) indicate that lack of noxin increases the rate of programmed death in cells under normal conditions or when challenged with stressful stimuli.

### Example 19

### Cell Death is Increased by Noxin Gene Ablation or Down Regulation—RNAi Experiment

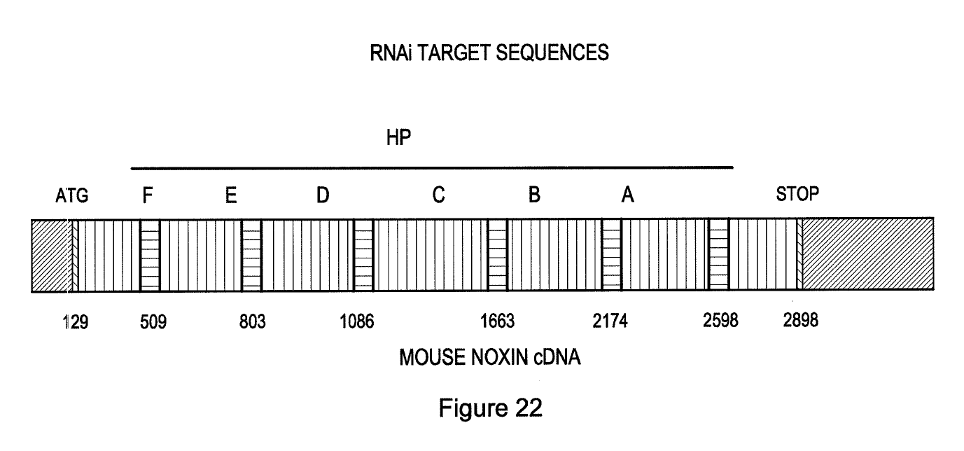

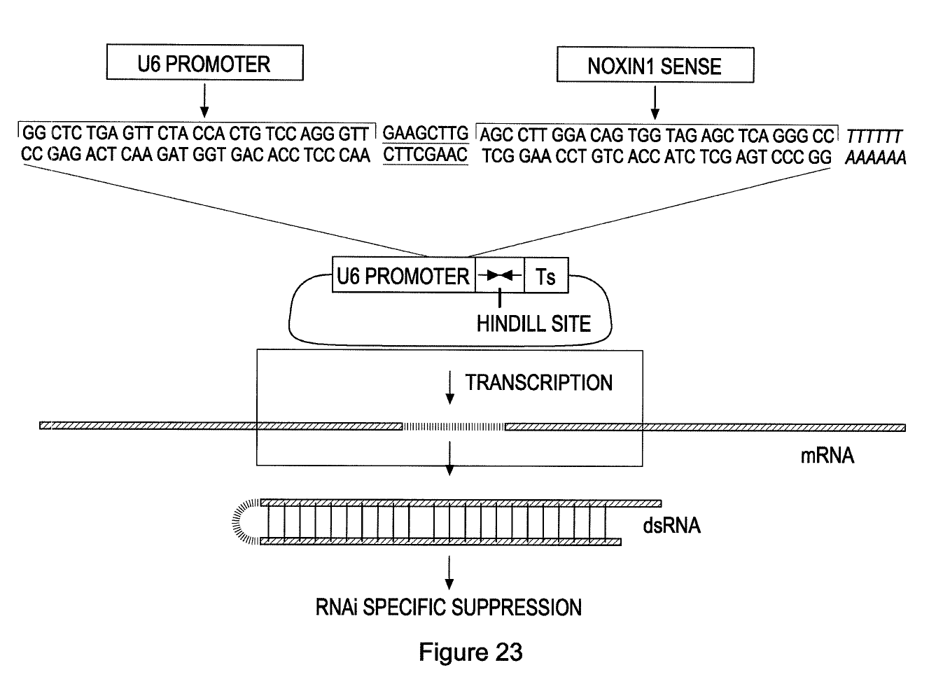

The role of noxin in normal cells was probed by down regulating its expression using the RNAi approach. Plasmids expressing six different noxin shRNA (FIG. 22, depicted as HPA-HPF) under control of the human U6 promoter were generated as described (Paddison et al., Proc Natl Acad Sci U.S.A. 99:1443-8 (2002)). The 6 hairpin structures correspond to the following noxin nucleotide positions:

HPA: nt 2598-2626 (SEQ ID NO: 14)

HPB: nt 2174-2202 (SEQ ID NO: 15)

HPC: nt 1163-1191 (SEQ ID NO: 16)

HPD: nt 1086-1114 (SEQ ID NO: 17)

HPE: nt 803-831 (SEQ ID NO: 18)

HPF: nt 509-537 (SEQ ID NO: 19)

For reference, the start codon is at nt 129 and the stop codon is at nt 2898.

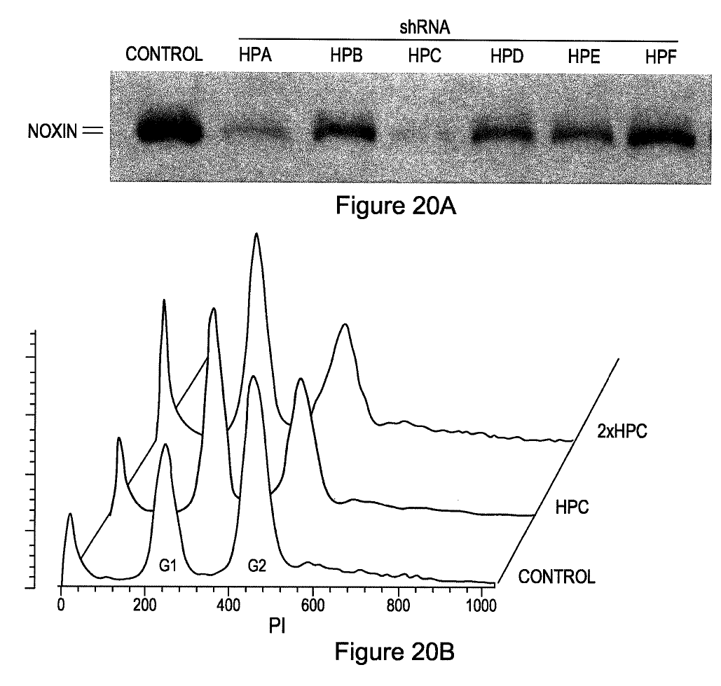

The target U6 promoter cassette DNA was amplified using a 5′-primer corresponding to the SP6 site at the 5′-end of the U6 promoter cassette and a long 3′ primer complementary to the 3′-end of the promoter and containing sequences for the noxin shRNA and a pol III termination site. The PCR fragments were subcloned into pENTR-TOPO-D vector (Invitrogen) and then transferred into pMSCV-puro vector by a clonase (Invitrogen) reaction. These six, short hairpin RNA (shRNA) constructs were generated and corresponded to different regions of noxin mRNA and their binding efficiency was compared by co-expressing them with recombinant noxin cDNA (FIG. 20A). To select shRNA constructs, six different shRNA constructs (HPA to HPF) were produced and examined for their ability to suppress the expression of noxin protein. NIH3T3 cells were co-transfected with constructs coding for noxin shRNAs, FLAG-noxin, and luciferase. Lysates were examined for luciferase expression. After normalization, protein samples were analyzed for the expression of FLAG-noxin protein by Western blot. HPC hairpin construct showed the strongest inhibition of noxin expression, whereas HPF had no effect and was used as a control. The shRNA construct that showed the highest efficiency in this assay (HPC) was used to transfect NIH3T3 cells treated with nocodazole (to arrest the cells in the G₂/M phase).

Panel 20B shows that nocodazole treatment resulted in accumulation of cells in the G₂/M phase and that transfection with HPC decreased the fraction of cells in G₂/M phase, while increasing the sub-G₁ population. For this experiment, NIH3T3 cells were transfected with 1 μg (HPC) or 2 μg (2×HPC) of the HPC shRNA construct, treated with nocodazole for 20 h, collected, stained with PI, and analyzed for DNA content using flow cytometry. The downregulation of noxin using this shRNA construct decreased the fraction of cells in G₂/M and increased the fraction of cells in the sub-G₁ population: 5.7±0.4% in control cells vs. 7.8±1.3% in cells transfected with 1 μg of HPC, and 8.0±1.7% in cells transfected with 2 μg of HPC (FIG. 20B).

20A: Western blot image showing that noxin knockdown by small hairpin RNA interference molecules (shRNA) increased the fraction of apoptotic cells after treatment with nocodazole.

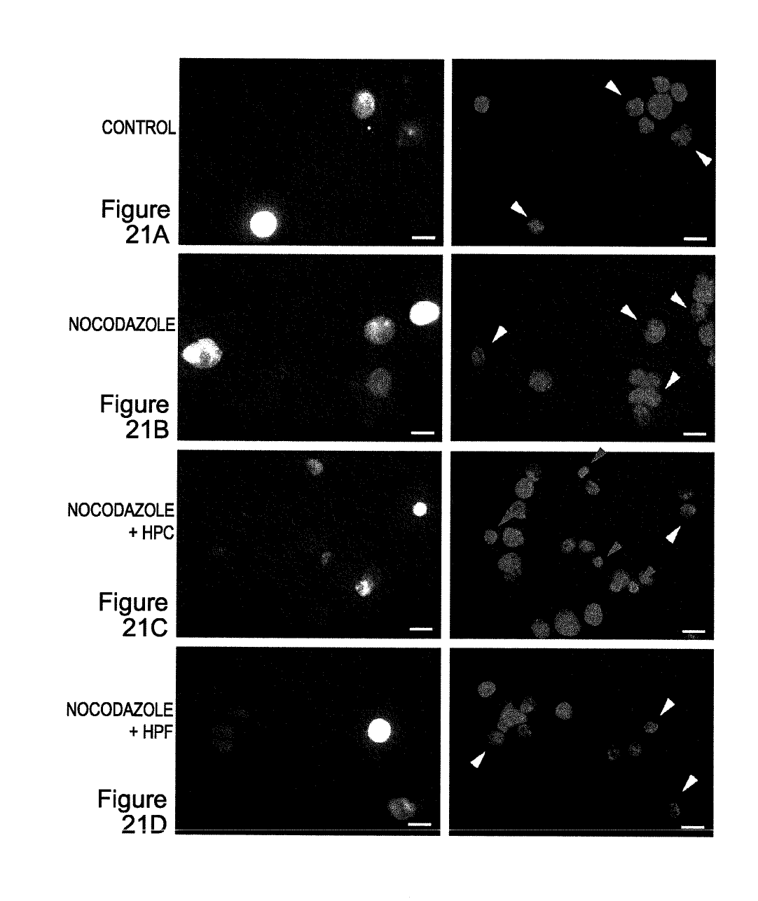

Cells transfected with HPC often showed condensed nuclei characteristic of apoptotic cells (FIG. 21). Panels 21A-D are images of cells that were co-transfected with the HPC or HPF shRNA constructs together with YFP-actin construct and stained with Hoechst. shRNA-transfected cells were identified by the YFP expression. HPC-transfected cells (yellow) showed condensed chromatin structures, corresponding to apoptotic cells (red arrowheads). The nuclei of cells transfected with HPF and YFP-actin or with YFP-actin alone and the nuclei of non-transfected cells had normal shape (light blue arrowheads). Scale bar: 10 μm.

A decrease or loss of noxin leads to increased cell death, as evidenced by the results of three different types of assays (TUNEL, flow cytometry, and annexin V/PI staining) and in all tested settings, whether using cells from wild type or noxin knockout animals or cells with RNAi-induced downregulation of noxin expression (FIGS. 17-21). Noxin is further considered to be part of stress response, based on the presence, in the noxin molecule, of phosphorylation sites for the ATM, DNA-PK, and Akt kinases, each of which plays a critical role in the stress response (Harris et al., Oncogene 24:2899-908 (2005); Kastan et al., Nature 432:316-323 (2004); Poyurovsky et al., Genes Development 20:125-131 (2006); Zhou et al., Nature 408:433-9 (2000)). This link is also supported by the fact that for each stress stimulus, induction of noxin was dependent on p53 (FIG. 6D). Furthermore, noxin's participation in the stress response is compatible with the finding that it accumulates in the nucleus in response to stress. At the same time, since it takes several hours after the introduction of the stressor for the full induction of noxin and since this induction is dependent on p53, it is conceivable that noxin does not participate in the first line of defense against stress (e.g., as compared with p53 whose rapid involvement in the repair process is achieved via posttranslational modification and increased stability of preexisting protein stores) (Harris et al, and Poyurovsky et al., see above.).

The following references, and any references that appear in the specification, are incorporated by reference in their entirety.
1. Bakkenist, C. J., and M. B. Kastan. 2004. Initiating cellular stress responses. Cell 118:9-17.
2. Bartek, J., and J. Lukas. 2001. Mammalian G1- and S-phase checkpoints in response to DNA damage. Curr. Opin. Cell Biol. 13:738-47.
3. Boehning, D., and S. H. Snyder. 2003. Novel neural modulators. Annu. Rev. Neurosci. 26:105-31.
4. Brüne, B. 2003. Nitric oxide: NO apoptosis or turning it ON? Cell Death Differ. 10: 864-869.
5. Contestabile, A., and E. Ciani. 2004. Role of nitric oxide in the regulation of neuronal proliferation, survival and differentiation. Neurochem. Int. 45:903-14.
6. Decimo, D., et al. 1995. In situ hybridization of nucleic acid probes to cellular RNA, p. 183-210. In B. D. Hames, and S. J. Higgins (ed.), Gene Probes 2: A Practical Approach, vol. 2. IRL Press at Oxford University Press, New York.
7. Di Meglio, et al. 2004. Dual role for poly(ADP-ribose)polymerase-1 and -2 and poly(ADP-ribose)glycohydrolase as DNA-repair and pro-apoptotic factors in rat germinal cells exposed to nitric oxide donors. Biochim. Biophys. Acta 1692:35-44.
8. Enikolopov, G., et al. 1999. Nitric oxide and Drosophila development. Cell Death Differ 6:956-63.
9. Estrada, C., and M. Murillo-Carretero. 2005. Nitric oxide and adult neurogenesis in health and disease. Neuroscientist 11:294-307.
10. Gibbs, S. M. 2003. Regulation of neuronal proliferation and differentiation by nitric oxide. Mol. Neurobiol. 27:107-20.
11. Gudkov, A. V., and E. A. Komarova. 2003. The role of p53 in determining sensitivity to radiotherapy. Nat. Rev. Cancer 3:117-29.
12. Harris, S. L., and A. J. Levine. 2005. The p53 pathway: positive and negative feedback loops. Oncogene 24:2899-908.
13. Hemish, J., et al. 2003. Nitric oxide activates diverse signaling pathways to regulate gene expression. J. Biol. Chem. 278:42321-9.
14. Hofseth, L. et al. 2003. Nitric oxide-induced cellular stress and p53 activation in chronic inflammation. Proc. Nat. Acad. Sci. USA 100:143-8.
15. Ignarro, L. J. 2000. Nitric Oxide: Biology and Pathobiology. Academic press, San Diego.
16. Kastan, M. B., and J. Bartek. 2004. Cell-cycle checkpoints and cancer. Nature 432:316-23.
17. Li, C.-Q., and G. N. Wogan. 2005. Nitric oxide as a modulator of apoptosis. Cancer Let. 226:1-15.
18. McLaughlin, L. M., and B. Demple. 2005. Nitric oxide-induced apoptosis in lymphoblastoid and fibroblast cells dependent on the phosphorylation and activation of p53. Cancer Res. 65:6097-104.
19. Nathan, C. 2003. Specificity of a third kind: reactive oxygen and nitrogen intermediates in cell signaling. J. Clin. Invest. 111:769-78.
20. Packer, M. A. et al. 2003. Nitric oxide negatively regulates mammalian adult neurogenesis. Proc. Nat. Acad. Sci. USA 100:9566-71.
21. Paddison, P. J., et al. 2002. Stable suppression of gene expression by RNAi in mammalian cells. Proc. Nat. Acad. Sci. USA 99:1443-8.
22. Peunova, N., and G. Enikolopov. 1995. Nitric oxide triggers a switch to growth arrest during differentiation of neuronal cells. Nature 375:68-73.
23. Poyurovsky, M. V., and C. Prives. 2006. Unleashing the power of p53: lessons from mice and men. Genes Dev. 20:125-31.
24. Sharma, R. V., et al. 1999. NOS gene transfer inhibits expression of cell cycle regulatory molecules in vascular smooth muscle cells. Am. J. Physiol. 276:H1450-9.
25. Tanner, F. C., et al. 2000. Nitric oxide modulates expression of cell cycle regulatory proteins: a cytostatic strategy for inhibition of human vascular smooth muscle cell proliferation. Circulation 101:1982-9.
26. Zhou, B. B., and S. J. Elledge. 2000. The DNA damage response: putting checkpoints perspective. Nature 408:433-9.

1. An isolated polynucleotide encoding a noxin polypeptide, or a fragment thereof.

2. The polynucleotide according to claim 1, wherein the noxin polypeptide is selected from the group consisting of human noxin, mouse noxin, and rat noxin.

3. The polynucleotide according to claim 1, wherein the noxin polypeptide has an amino acid sequence selected from the group consisting of SEQ ID NO: 2, SEQ ID NO: 4 and SEQ ID NO: 6, and variants thereof.

4. The polynucleotide according to claim 1, wherein the polynucleotide has the nucleotide sequence selected from the group consisting of SEQ ID NO: 1, SEQ NO: 3, and SEQ ID NO: 5 and variants thereof.

5.-8. (canceled)

9. The vector according to claim 7, wherein the vector further comprises a second polynucleotide having an additional coding sequence fused in-frame with a noxin coding sequence.

10. (canceled)

11. A vector comprising a disrupted noxin gene.

12.-13. (canceled)

14. A non-human transgenic animal whose genome comprises a disrupted endogenous noxin gene, wherein said disruption results in said animal exhibiting decreased noxin protein expression as compared to a wild-type animal or no noxin protein expression.

15. (canceled)

16. The transgenic animal of claim 14, wherein said disruption abolishes noxin expression.

17. The transgenic animal of any of claim 14, wherein the animal is a rodent.

18.-21. (canceled)

22. An isolated nucleic acid comprising a noxin knockout construct comprising a selectable marker sequence flanked by DNA sequences homologous to the endogenous noxin gene, wherein when said construct is introduced into a non-human animal or an ancestor of said animal at an embryonic stage, said selectable marker sequence disrupts the endogenous noxin gene in the genome of said animal such that said animal exhibits decreased noxin production as compared to a wild type animal.

23. A mouse cell line comprising the noxin knockout construct of claim 22.

24. (canceled)

25. A method for protecting a cell from stress damage comprising the step of enhancing the expression of noxin in said cell.

26. The method according to claim 25 wherein noxin expression is enhanced by introduction of an exogenous noxin gene, operably linked to a promoter functional in the cell, into the cell.

27. (canceled)

28. A method of decreasing the occurrence of cell death or preventing cell death caused by stress comprising the step of enhancing expression of the noxin gene in cells subjected to cell-death inducing stimuli.

29. The method according to claim 28 wherein noxin expression is enhanced by introduction of an exogenous noxin gene, operably linked to a promoter functional in the cell, into the cell, and optionally inducing the noxin expression.

30. (canceled)

31. A method of inducing cell death comprising the step of reducing noxin activity in the cell.

32. The method according to claim 31, wherein the noxin activity is reduced by inhibiting or reducing noxin transcription in the cell.

33.-34. (canceled)

35. The method according to claim 34, wherein the noxin translation is inhibited by RNA interference (RNAi).

36.-37. (canceled)

38. A method of inducing cell cycle arrest in a cell comprising the step of increasing noxin activity in the cell.

39. (canceled)

40. The method according to claim 38, wherein noxin expression is enhanced by introduction of an exogenous noxin gene, operably linked to a promoter functional in the cell, into the cell, and optionally inducing the noxin expression.

41. A method of preventing cell cycle arrest of a cell comprising the step of reducing or inhibiting noxin activity in the cell.

42. The method according to claim 41, wherein the noxin activity is reduced by inhibiting or reducing noxin transcription in the cell.

43.-44. (canceled)

45. The method according to claim 44, wherein the noxin translation is inhibited by RNA interference (RNAi).

46.-47. (canceled)

48. A method of assessing genome damage by determining the expression of noxin comprising the steps of detecting noxin expression by measuring:
(a) noxin mRNA by Northern blot analysis;
(b) noxin mRNA by Q-PCR; or
(c) noxin polypeptide by western blot.

49. A method of identifying an agent that controls noxin expression comprising the steps of:
(a) exposing a cell to a candidate agent;
(b) measuring the expression level of noxin in the cell and in control cells not exposed to the candidate agent, by measuring:
(i) noxin mRNA by Northern blot analysis;
(ii) noxin mRNA by Q-PCR; or
(iii) noxin polypeptide by western blot;
wherein an agent that increases noxin expression over the control is identified.

50. A method of identifying an agent that affects cellular response to a stress factor comprising the steps of:
(a) exposing a cell to a stress-inducing condition;
(b) exposing the cell to a candidate agent;
(b) measuring the expression level of noxin by detecting noxin expression in the cell and in control cells not exposed to the candidate agent, by measuring:
(i) noxin mRNA by Northern blot analysis;
(ii) noxin mRNA by Q-PCR; or
(iii) noxin polypeptide by western blot;
wherein an agent that increases noxin expression over the control is identified.

51. A method of identifying an agent that protects cells from stress induced damages comprising the steps of:
(a) exposing null noxin cells derived from non-human noxin^{-/-} transgenic animal to stress inducing conditions in the presence or absence of a candidate agent; and
(b) measuring the percentage of cells entering apoptosis;
wherein an agent that prevents apoptosis over the control is identified.


SEQUENCE LISTING (21 sequences)

SEQ ID NO: 1 - DNA, 3384 bp, Mus sp
cctgttggcctactggagattcgattgctggcgtttggagagcggcggaccgagcggccggagggcggagcggttgcccgggcttgtctgtgtcctttgcctcagataaaattttaagtgcctccattatgccaagaagggccaggttttggatgacttcaactaacagtagagattgctgtcctagggacaaagtatagacacatgaataaaagacggaaattcctttttgcctcagtacttgctgtccagaattcgaattttatatatccatcatgtcaaaggtgcttctctaagataatccttgagtccaaaaggtttacttgtcccaaatgtggctcttctggtgacactggaagtaccaattacagatacaaactgtccttgaaagtggcagaatccagcaaactatttttcattactgtatttgggagctgcttagatacattttttggacttacggccactggtttacacaggtaccttgaagattctattaaaaacctagaaaccctggacagtggtagaactcagagcctactgaccacagcagtagaaaagtgttttgttggacaaagctttgtatttggagtaacgaattttggagatgtatgtggacatgattcagattccagtaactttcagcagccttgctgtaagcacagaggagaagtcaggacacttgtagcttcccagattattctgcctagcccacatgttaaaggatttacagtcattgcctatttgcatccgcttttacacaagaaacctcactgtggctcccaggaacatagcagccagtcactcacttcagatgactcagacagtgatcttaacaacatacagggctctgggaatacatcctggttttcagagtctagtgtcggagaggattttttcagatactgggagccatctcttgaactgacttcttctgactcacaagtaacatctagtgatgactttccagcatcaaaacaaatcatggccagtggtactcctaaccaaaacagacactgcatctccgtttccgaggtcactagttctaagaactgccatgactcccttcagagtttatggagccttcattcttgtatggacaagaataatacaacaggaaagttgggtgaagaattaggcttaccgagtccagtttgcaacagttgtcatgaaagtagattgtctgactctcattttttcccttcacaaatgcaagaacccttcgaggaacataatttagaatgctatagtaaagcagaaaaaaatgattattcccagtatgatatcgcatgttaccagcatcatgaagtaaatactacccccatccttcaacagaggtcttctgcattttcattgtcatcactgaaacccgaagaaacagcgaatccttcccagaactctgactccttgatttgggatgatctaccactctctgaaagtctgaacaagtttctagcagttgttgaaagtgaggttgctgtaactgagatagatgctaagaacagaaaacagggtatggataaaagcattgataaatgtcacaaaaactacagcagattatcactgactccatggagaaatactagagccttaaatacatcatgtttcagcttaagatcatcacaagcaatgatgaaagactctgggaaagaggccctcttttccaactgtaagtctaattcaagtcctcacattcagaaagaatcgaaagcagacaagacagaagcggctgtctccatagccggtagtagaagtgatatctctgaggattttctaccggatacttgctcatcagcactgtttacatcttcaaaagatagggaagccttcattacccaaaagaggactagtggggtcttgcaacaaaggaatgaaatttcatgcaggcccagtacctcaataagtgactgttctgatctcaggagcagatattttactggatgtggacaaaaattacattcaggaacaaaaggaaaattggcaatacaaaactgttctaaaaaatacagcgatgcttctgatcttcacaaattagaaaataagcatagatggccgaaaaaggaagatgacaatttcacaatatgcagaaaactcacatatcctttagagaccctttgcagtagcccagaaagtataaatacatcgaaagagatgccttacaggcctagcaataataacttaacaccaagttcttctggtgatcatgaaggtagttacaatgcttctgctggtctttttgattatattgctaaagataaggacattggaacagaaattactaaaataccacaggataacttattaccactggaagtaccttgcacagaaaattatcctataaatgagaaccgcggccagccttcacaaaaaccatccttgcaaagcatatcaccctcaaggtattcaagaccaaggtctcagtcagactcagaatgtgattttgaagaaagccaagactttgttccatgttcacagtcaactccagttgcaggcttccaccaaagaattcatggcttaaatggtgcttccaaaatactgccttccatatattcaaatcataatgctaattataaaaacagaaaaaattctcctctaactggccagtatcaagccacctcagcctgctcaaagaatgtaaaagcatttcggcagaaacccgaaagtccttttgtgtctagtttgacacaagcaaatgttttcaaccactgccctgctgctgagagccttggaaatgatgtggatgaatgggtcccccctaccaccaagaaagtatttatttcagatattcttggattcagagtcatgggtttaagaaaatgccttgatctccattattcccctgacccaaaagagttaccaaggaagaaactgataaaagtcacacataaaaccaacatttaattaagaagttaaagactatacctatagttgcaaaaccaaaagtgctcaaagcatgactataaatgatcaggctgaaaataggaaaagcccagttggggatggcttttcagaataatacttttagtgtctgatacaaaaagcacttagtaactgaaggtattttcatataaagtcacatgaacccggttcccagaaattttaagtgatggcagatctgtttggaaatgtttgctttcactgatacagagagcagtctttatgttttgaagacttacaggaagtcgttaatagtggactgtactgaatggatctttcaaggtttccaccatggctttctttttttctccagaatactgtgattaagataactgagcacttctccttacactgactgtatgaatatgttgagtttgtatttaaaagtatttgttaaaatatagacatccagaagtaaagtctttacaatccaaaaaaaaaaaaaaa

SEQ ID NO: 2 - protein, 898 aa, Mus sp
MNKRRKFLFASVLAVQNSNFIYPSCQRCFSKIILESKRFTCPKCGSSGDTGSTNYRYKLSLKVAESSKLFFITVFGSCLDTFFGLTATGLHRYLEDSIKNLETLDSGRTQSLLTTAVEKCFVGQSFVFGVTNFGDVCGHDSDSSNFQQPCCKHRGEVRTLVASQIILPSPHVKGFTVIAYLHPLLHKKPHCGSQEHSSQSLTSDDSDSDLNNIQGSGNTSWFSESSVGEDFFRYWEPSLELTSSDSQVTSSDDFPASKQIMASGTPNQNRHCISVSEVTSSKNCHDSLQSLWSLHSCMDKNNTTGKLGEELGLPSPVCNSCHESRLSDSHFFPSQMQEPFEEHNLECYSKAEKNDYSQYDIACYQHHEVNTTPILQQRSSAFSLSSLKPEETANPSQNSDSLIWDDLPLSESLNKFLAVVESEVAVTEIDAKNRKQGMDKSIDKCHKNYSRLSLTPWRNTRALNTSCFSLRSSQAMMKDSGKEALFSNCKSNSSPHIQKESKADKTEAAVSIAGSRSDISEDFLPDTCSSALFTSSKDREAFITQKRTSGVLQQRNEISCRPSTSISDCSDLRSRYFTGCGQKLHSGTKGKLAIQNCSKKYSDASDLHKLENKHRWPKKEDDNFTICRKLTYPLETLCSSPESINTSKEMPYRPSNNNLTPSSSGDHEGSYNASAGLFDYIAKDKDIGTEITKIPQDNLLPLEVPCTENYPINENRGQPSQKPSLQSISPSRYSRPRSQSDSECDFEESQDFVPCSQSTPVAGFHQRIHGLNGASKILPSIYSNHNANYKNRKNSPLTGQYQATSACSKNVKAFRQKPESPFVSSLTQANVFNHCPAAESLGNDVDEWVPPTTKKVFISDILGFRVMGLRKCLDLHYSPDPKELPRKKLIKVTHKTNI

SEQ ID NO: 3 - DNA, 3545 bp, Homo sapiens
gggagattcgattggaaggctgccggcgtgctactgagttcggccggtccgagtcactgtgcgtcgcctgggcgcgttcctggtcttctcccccagggtcttgctctgtcacccaggctggagtgccatggcatcatcatagctcactatggccttgatcctcctgccttagcctcccaagcagctggtattacagaccacggtgtgaacacatgaacagaagacgaaaatttcttctagcctcagtacttgctctccagaattcaagttttatatatccatcatgtcagaagtgcttctctaggataatcctggtctccaaaaggtctaattgtccaaaatgtggctctactggtgaatctggaaatgccaattacagatacaaactttccttaaaagttgcagaatcaaacaaattgtttgttattactgtatttggaagttgcttagatacattttttggtcttactgccactggtttgcacaggtacattcaggatcctaataaaattccagaaacactggacaatgatacaactcagaatctattaactaaagcagttgaaacttgctttgttggacaaagctttatttttggagtgacgaattttgaaaaccaacctggacaaggttcagatgccagtaacttcttacagcaatgctctgaccacaaaagaaaagccaaagcactagtggcttgccagattgttctaccagacccaggtattgcaggctttactgtcattgactacttccatcaacttttgcagacttttaatttcaggaaacttcagtgtgactctcaggcacctaacaatcacttacttgctttagatcactcaaatagtgatctcagcagcacatatacttctgacagcacttctgattttttcaagtcctgcagcaaggatactttttcaaaattctggcagccatcacttgaattcacttgcattgtttcacaactaacagataatgatgatttttcagcttcagaacaaagtaaggcctttggtactcttcagcagaacagaaagtccatctccattgcagaggccactggttccagtagctgccatgatcccattcaggattcatggagccttgtttcatatatggataaaaagagtacagcagaaaagttgggtaaagaacttggcttacaagctaaggagctgagtgcagttcacagcagtcatcatgaaattggagttaatgactctaatttattctctttggaaatgcgagagccccttgagtcaagtaatacaaaatccttccacagtgcagtggaaattaaaaataggtcccagcatgagctaccatgttttcagcatcatggtatagataccccaactagccttcagaagagatctgcatgttgtccaccttcgttactcagacttgaagagacagccagcagttcccaggatggtgaccctcaaatttgggatgatctgccattctctgaaagcctgaacaagtttctggcagttcttgaaagtgagattgctgtaacccaggcagatgtcagtagtaggaaacatcatgtagataatgacattgataaatttcatgcagaccacagcagtttatctgtgactccccagagaactactggagccctgcatacaccacctatagctttaagatcatcacaagtaatagtcaaagcaaactgtagcaaagatgacttccttttcaactgtaaaggaaatctaagtcctagtgttgaaaaggagtcacaaccagataacaaagtagaggctgtctctgtaaatcataatggaagagatatgtcagaatattttttaccgaatccttacctgtcagctctgtcttcatcttcaaaagatttagaaacaatagttactcttaagaagactatcagaatctcaccacacagggagagtgaccattctagtctaaataacaaatatttgaatggatgtggagaaatatcagtttcagaaatgaatgaaaagttgacaactctgtgttataggaagtataatgatgtctctgatctttgcaaattagaaaataaacaatattgtaggtggtccaagaaccaagatgacagttttacaatttgcaggaaacttacatatcctttagaaactctttgcaatagtccaaatagaagtacaaatacattgaaagaaatgccttggggacatatcaataacaacgtaacacagagctattctattggttatgaaggtagctatgatgcctctgctgatctctttgatgatattgctaaagaaatggacattgcaactgagattaccaaaaaatcacaggatattttgttaaaatggggaacatctttggcagaaagtcacccttcagagtctgatttttcactgagatcactttctgaagacttcatccagccttcacaaaaattatccttgcaaagcctatctgactctaggcattcaagaacatgctctccaacacctcattttcaatcagattcagaatataattttgaaaatagtcaagactttgttccatgttcacagtcaactccaatttcagggttccaccaaacaagaattcatgggataaacagagctttcaaaaaacctgtattttattcagatcttgatggtaactatgaaaaaataaggattttccctgaaaatgacaaacagcaagccagcccaagctgtccaaaaaatataaaaacacctagccagaaaatcagaagccctattgtatctggtatttcacaaccagacgttttcaatcactacccttttgctgagtgccatgaaactgatagtgatgaatgggtccctcctaccacacaaaaaatatttccttcagatatgcttggattccaaggcataggtctagggaaatgccttgctgcctatcatttccctgatcaacaagagttaccaagaaagaaactgaaacatattagacaaggaaccaataaaggtttaattaagaagaaattaaagaatatgcttgcagcagttgttacgaaaaagaaaactcataaatataactgtaaaagttcaggctggatttccaaatgtccagacattcaagtcttagcagcacctcagctgcaccctattcttggacctgattcttgttcagaagtcaaatgttgccttccattttcagaaaaaggcccaccttcagtgtgtgaaactcgaagtgcttggtcacctgaattgttttcataaaaagtcacctgaacccaattcctgaacttttaaatctgtttggaaatgtttgccttcaggggtacggaaagcattctttacattttgaacacttggagagaagcaaattgaaaacaggactctgctgggagctactgtgccttttaaaatataaagccattgttttccccagggttttatctagaatactatgattaggtagttgagcactttatcttatactgtttattgtactttaataatattgttaagattgtttttgaaagtattaatgtttgttaaaatcacatatacatccagaaataaagactttgcaaaccaaaaaaaaaaaaaaaa

SEQ ID NO: 4 - protein, 914 aa, Homo sapiens
MNRRRKFLLASVLALQNSSFIYPSCQKCFSRIILVSKRSNCPKCGSTGESGNANYRYKLSLKVAESNKLFVITVFGSCLDTFFGLTATGLHRYIQDPNKIPETLDNDTTQNLLTKAVETCFVGQSFIFGVTNFENQPGQGSDASNFLQQCSDHKRKAKALVACQIVLPDPGIAGFTVIDYFHQLLQTFNFRKLQCDSQAPNNHLLALDHSNSDLSSIYTSDSTSDFFKSCSKDTFSKFWQPSLEFTCIVSQLTDNDDFSASEQSKASGTLQQNRKSISIAEATGSSSCHDPIQDSWSLVSYMDKKSTAEKLGKELGLQAKELSAVHSSHHEIGVNDSNLFSLEMREPLESSNTKSFHSAVEIKNRSQHELPCFQHHGIDTPTSLQKRSACCPPSLLRLEETASSSQDGDPQIWDDLPFSESLNKFLAVLESEIAVTQADVSSRKHHVDNDIDKFHADHSSLSVTPQRTTGALHTPPIALRSSQVIVKANCSKDDFLFNCKGNLSPSVEKESQPDNKVEAVSVNHNGRDMSEYFLPNPYLSALSSSSKDLETIVTLKKTIRISPHRESDHSSLNNKYLNGCGEISVSEMNEKLTTLCYRKYNDVSDLCKLENKQYCRWSKNQDDSFTICRKLTYPLETLCNSPNRSTNTLKEMPWGHINNNVTQSYSIGYEGSYDASADLFDDIAKEMDIATEITKKSQDILLKWGTSLAESHPSESDFSLRSLSEDFIQPSQKLSLQSLSDSRHSRTCSPTPHFQSDSEYNFESSQDFVPCSQSTPISGFHQTRIHGINRAFKKPVFYSDLDGNYEKIRIFPENDKQQASPSCSDASNFLQQCSDHKRKAGISQPDVFNHYPFAECHETDSDEWVPPTTQKIFPSDMLGFQGIGLGKCLAAYHFPDQQELPRKKLKHIRQGTNK

SEQ ID NO: 5 - DNA, 3318 bp, Rattus sp
cggagttccttcatggttgcccgggcttgcctgggtcctttgcttcagataaaattttaagtgcctcccattatgccaagaagggccaggttttggatgacttcaattaccaatagagactgctgtcctagggacaagatagaggcacatgaatagaagacggaaattcctttttgcctcagtacttgctctccagaattcgagttttatatatccatcatgtcaaaggtgcttctccaggatattccttgagtccaaaaggtttacttgtcccaagtgtggctgtaccggtgatgctgagagtgccaactacagatatagactgtccttgaaagtggcagagtccaacacgttatttgtcattactgtgtttgggagctgcttagatacttattttggactcactgctactggtttacacaggtaccttaaggattctagtaaaatcccagaaaccctggacagtggtagaactcagagcctgttgaccacagcagtagaaaagtgttttgttggacaaagctttgtgtttggagtaacgaactttggagatgtatgtggacaccattccaattgcagtaacttccagcagccttgctgtaagcacagaggagaagtcaggacactcgtagcctcccagatcgtcctgcctgacccgcgtgttacaggcttcactgtcattgactatttacatccgcttttacacaggaaacatcactgtgactctcaggaccatagcagccagtcactcacttcagatcactcagacagcgagttcaacagcatacagggctctgggaatacatcctgggttttggagtctagcagagaggactttttcagattctggcagccatctcttgagctgacgtctactgattcacacataacatctaatgatgacttcccaccttcagaacagctcgtggccagtggtactcctacccaaaacggacactgcgtctccttctctgaggtcactagttctaacaactgccacgattccctccagagtttatggagctttgtttcttgtatggacaagaataatacaacaggaaagttgggtgaagaattaggtttactgagtccaatttgcagcagttgtcatgaaagtagactgactgactctaattttttcccttcacaaatgcaaaaaccctttaaggaacataatttagaatgctatagtaaagcagaaaaaaatgattactcccagtatgatatcccatgttaccagcatcgtgaagtaaataccaccattcttcaacagggatcttctgcattttcattgtcatcactgaaacccgaagaaacagcgagtgcttcccagaagtctgactccttgatttgggatgatataccattctctgaaagcctgaacaagtttctagcagttgttgaaagtgagattgctgtagctgagatagatactcagaatagaaagcagggtacagataacagcactgataaatgccacaaaaactacagcagattatcattgactcctctgagagatactagagccttaaatacgccgcctttcggcttaaggtcatcacaagcaatgagagaaaactcaaggagagaggccttcttttataacagtaagtctagttcaagttctcaaatccaaagagaatcacatccagacaaggcagcagcggctgtctctataggcagtaataggagcgatatctccaaggattttctaccagatacttgttcatcagctctgtttacatcttcagatgacatggaagcctccattagccaaaagaggactagtggggtcctgcaacaaaggaatgaaatttcgtgcaggctcagaacctcaataagtgactgttctgatctcagtaacagctgttttactggatgtgaagaaaaatcgcattcaggaacaaaagaaaaattggcaatacaaaactgttctaaaaaatacaatgatgtttctgatcttcacaaattagaaaataagcggagttatagatggccggaaaagcaagatgacaatttcacaattagccggaaactcacatatcctttagagacctttcgcgggagcccaaaaagcatatcaaaagaaaagccttataggcctagcaataataacttaacacaaagttcttctgctgatcatgaaggcagctacaatgcttctgctgatctttttgatgattttgctaaagataaggacactgaaacagaaattaccaaactaccgcaagatattttgttaccactggaagcatcttgcacagaaaattatcctataaatgagaactgcaaccatccttcacaaaaactctccttgcaaagcatatcaccctcagagtatcaaagaccaagctctcagtcagactcagaatgtgattttgaagaaagccaagaagactttgttccatgttcacagtcaactccagttgcaggcttccaccaaagaattcatggcttaaatggtgcttccaaaatactgccttccttctattcacatcttaatgctaattataaaaacacaaacatttctcctctaactggcaaatgtcaagccaccccaacctgcccaaagaatgtaaaaacatctacgcagaagctcaaccactgccctgctgctgagtgccttgaaaatgatatggatgaatgggtcccccctactactaaaaaagtatttatttcagatgtttgtggattgagagtcacgtgtttaagaaaatgccttgctgtccattattcccctgaccaaaaagagttaccaaggaagaaactgataaaagtcacacataaaaccaacaaataattaagaagttaaagactatattttgtaccaataattacaaaaccaaaagtgtcttaagtgtgattgtaaatgatcaggctgaaagtaggaaaagtccatttggggatggcctttcaggagtcaagtggaagtcatcttcccttttcataatacttcagtgtctgataaaaaaagtacttggtttactcaaagtatttccatataaagtcataggaatccagttcccagaaattttaagcaatggcaaatctatttggaaatttttgctttcactgatacagagagcagtctatattttgaacacttacagaaagtcattaatagaaaatggactgtactgtgtgctgttggatctttcaagattttcaccatggctttttttttcctccagaatactgtaatttagataactgagcacttttccttacattgactgtattaatatgttggttttgtatatttaaaagtatttgttaaaatatagacatacagaaataaagtctttacaaattaaaata

SEQ ID NO: 6 - protein, 883 aa, Rattus sp
MNRRRKFLFASVLALQNSSFIYPSCQRCFSRIFLESKRFTCPKCGCTGDAESANYRYRLSLKVAESNTLFVITVFGSCLDTYFGLTATGLHRYLKDSSKIPETLDSGRTQSLLTTAVEKCFVGQSFVFGVTNFGDVCGHHSNCSNFQQPCCKHRGEVRTLVASQIVLPDPRVTGFTVIDYLHPLLHRKHHCDSQDHSSQSLTSDHSDSEFNSIQGSGNTSWVLESSREDFFRFWQPSLELTSTDSHITSNDDFPPSEQLVASGTPTQNGHCVSFSEVTSSNNCHDSLQSLWSFVSCMDKNNTTGKLGEELGLLSPICSSCHESRLTDSNFFPSQMQEPFKEHNLECYSKAEKNDYSQYDIPCYQHREVNTTILQQGSSAFSLSSLKPEETASASQKSDSLIWDDIPFSESLNKFLAVVESEIAVAEIDTQNRKQGTDNSTDKCHKNYSRLSLTPLRDTRALNTPPFGLRSSQAMRENSRREAFFYNSKSSSSSQIQRESHPDKAAAAVSIGSNRSDISKDFLPDTCSSALFTSSDDMEASISQKRTSGVLQQRNEISCRLRTSISDCSDLSNSCFTGCEEKSHSGTKEKLAIQNCSKKYNDVSDLHKLENKRSYRWPEKQDDNFTISRKLTYPLETFRGSPKSISKEKPYRPSNNNLTQSSSADHEGSYNASADLFDDFAKDKDTETEITKLPQDILLPLEASCTENYPINENCNHPSQKLSLQSISPSEYQRPSSQSDSECDFEESQEDFVPCSQSTPVAGFHQRIHGLNGASKILPSFYSHLNANYKNTNISPLTGKCQATPTCPKNVKTSTQKLNHCPAAECLENDMDEWVPPTTKKVFISDVCGLRVTCLRKCLAVHYSPDQKELPRKKLIKVTHKTNK

SEQ ID NO: 7 - DNA, 24 bp, Artificial sequence
  Synthetic oligonucleotide
tttctaccggatacttgctcatca

SEQ ID NO: 8 - DNA, 22 bp, Artificial sequence
  Synthetic oligonucleotide
gttgcaagaccccactagtcct

SEQ ID NO: 9 - DNA, 22 bp, Artificial sequence
  Synthetic oligonucleotide
cgtgaaaagatgacccagatca

SEQ ID NO: 10 - DNA, 21 bp, Artificial sequence
  Synthetic oligonucleotide
cacagcctggatggctacgta

SEQ ID NO: 11 - DNA, 22 bp, Artificial sequence
  Synthetic oligonucleotide
gaagcacctttgacatgatgga

SEQ ID NO: 12 - DNA, 20 bp, Artificial sequence
  Synthetic oligonucleotide
tggcccttcttggcataatg

SEQ ID NO: 13 - protein, 17 aa, Artificial sequence
  Mouse noxin amino acid 870-866
RKCLDLHYSPDPKELPR

SEQ ID NO: 14 - DNA, 29 bp, Artificial sequence
  Mouse noxin nucleotides 2598-2626
tggccagtatcaagccacctcagcctgct

SEQ ID NO: 15 - DNA, 29 bp, Artificial sequence
  Mouse noxin nucleotides 2174-2202
ataacttaacaccaagttcttctggtgat

SEQ ID NO: 16 - DNA, 29 bp, Artificial sequence
  Mouse noxin nucleotides 1163-1191
gttgtcatgaaagtagattgtctgactct

SEQ ID NO: 17 - DNA, 29 bp, Artificial sequence
  Mouse noxin nucleotides 1086-1114
tcattcttgtatggacaagaataatacaa

SEQ ID NO: 18 - DNA, 29 bp, Artificial sequence
  Mouse noxin nucleotides 803-831
cactcacttcagatgactcagacagtgat

SEQ ID NO: 19 - DNA, 29 bp, Artificial sequence
  Mouse noxin nucleotides 509-537
aaaccctggacagtggtagaactcagagc

SEQ ID NO: 20 - protein, 914 aa, Homo sapiens
MNRRRKFLLASVLALQNSSFIYPSCQKCFSRIILVSKRSNCPKCGSTGESGNANYRYKLSLKVAESNKLFVITVFGSCLDTFFGLTATGLHRYIQDPNKIPETLDNDTTQNLLTKAVETCFVGQSFIFGVTNFENQPGQGSDASNFLQQCSDHKRKAKALVACQIVLPDPGIAGFTVIDYFHQLLQTFNFRKLQCDSQAPNNHLLALDHSNSDLSSIYTSDSTSDFFKSCSKDTFSKFWQPSLEFTCIVSQLTDNDDFSASEQSKASGTLQQNRKSISIAEATGSSSCHDPIQDSWSLVSYMDKKSTAEKLGKELGLQAKELSAVHSSHHEIGVNDSNLFSLEMREPLESSNTKSFHSAVEIKNRSQHELPCFQHHGIDTPTSLQKRSACCPPSLLRLEETASSSQDGDPQIWDDLPFSESLNKFLAVLESEIAVTQADVSSRKHHVDNDIDKFHADHSSLSVTPQRTTGALHTPPIALRSSQVIVKANCSKDDFLFNCKGNLSPSVEKESQPDNKVEAVSVNHNGRDMSEYFLPNPYLSALSSSSKDLETIVTLKKTIRISPHRESDHSSLNNKYLNGCGEISVSEMNEKLTTLCYRKYNDVSDLCKLENKQYCRWSKNQDDSFTICRKLTYPLETLCNSPNRSTNTLKEMPWGHINNNVTQSYSIGYEGSYDASADLFDDIAKEMDIATEITKKSQDILLKWGTSLAESHPSESDFSLRSLSEDFIQPSQKLSLQSLSDSRHSRTCSPTPHFQSDSEYNFENSQDFVPCSQSTPISGFHQTRIHGINRAFKKPVFYSDLDGNYEKIRIFPENDKQQASPSCSDASNFLQQCSDHKRKAGISQPDVFNHYPFAECHETDSDEWVPPTTQKIFPSDMLGFQGIGLGKCLAAYHFPDQQELPRKKLKHIRQGTNK

SEQ ID NO: 21 - protein, 883 aa, Rattus sp
MNRRRKFLFASVLALQNSSFIYPSCQRCFSRIFLESKRFTCPKCGCTGDAESANYRYRLSLKVAESNTLFVITVFGSCLDTYFGLTATGLHRYLKDSSKIPETLDSGRTQSLLTTAVEKCFVGQSFVFGVTNFGDVCGHHSNCSNFQQPCCKHRGEVRTLVASQIVLPDPRVTGFTVIDYLHPLLHRKHHCDSQDHSSQSLTSDHSDSEFNSIQGSGNTSWVLESSREDFFRFWQPSLELTSTDSHITSNDDFPPSEQLVASGTPTQNGHCVSFSEVTSSNNCHDSLQSLWSFVSCMDKNNTTGKLGEELGLLSPICSSCHESRLTDSNFFPSQMQKPFKEHNLECYSKAEKNDYSQYDIPCYQHREVNTTILQQGSSAFSLSSLKPEETASASQKSDSLIWDDIPFSESLNKFLAVVESEIAVAEIDTQNRKQGTDNSTDKCHKNYSRLSLTPLRDTRALNTPPFGLRSSQAMRENSRREAFFYNSKSSSSSQIQRESHPDKAAAAVSIGSNRSDISKDFLPDTCSSALFTSSDDMEASISQKRTSGVLQQRNEISCRLRTSISDCSDLSNSCFTGCEEKSHSGTKEKLAIQNCSKKYNDVSDLHKLENKRSYRWPEKQDDNFTISRKLTYPLETFRGSPKSISKEKPYRPSNNNLTQSSSADHEGSYNASADLFDDFAKDKDTETEITKLPQDILLPLEASCTENYPINENCNHPSQKLSLQSISPSEYQRPSSQSDSECDFEESQEDFVPCSQSTPVAGFHQRIHGLNGASKILPSFYSHLNANYKNTNISPLTGKCQATPTCPKNVKTSTQKLNHCPAAECLENDMDEWVPPTTKKVFISDVCGLRVTCLRKCLAVHYSPDQKELPRKKLIKVTHKTNK